(19) United States
(12) Patent Application Publication
(10) Pub. No.: US 2021/0355479 A1
(43) Pub. Date: Nov. 18, 2021

(54) PARAMETERS FOR CONCENTRATION AND WASHING OF PARTICLES WITH ACOUSTICS

(71) Applicant: FloDesign Sonics, Inc., Wilbraham, MA (US)

(72) Inventors: Benjamin Ross-Johnsrud, Northampton, MA (US); Bart Lipkens, Bloomfield, CT (US)

(21) Appl. No.: 17/421,647

(22) PCT Filed: Jan. 21, 2020
(86) PCT No.: PCT/US2020/014492

§ 371 Date: Jul. 8, 2021

Related U.S. Application Data
(60) Provisional application No. 62/794,978, filed on Jan. 21, 2019.

(51) Int. Cl. C12N 13/00; C12M 1/00; B01D 21/28

(52) U.S. Cl. CPC C12N 13/00; B01D 21/283; C12M 47/02

(57) ABSTRACT

Multi-stage acoustophoretic devices for continuously separating a second fluid or a particulate from a host fluid are disclosed. Methods of operating the multi-stage acoustophoretic devices are also disclosed. The systems may include multiple acoustophoretic devices fluidly connected to one another in series, each acoustophoretic device comprising a flow chamber, an ultrasonic transducer capable of creating a multi-dimensional acoustic standing wave, and a reflector. The systems can further include pumps and flowmeters.

### CROSS-REFERENCE TO RELATED APPLICATIONS

The present application is a U.S. National Stage Application of PCT International Application No. PCT/US2020/014492, filed Jan. 21, 2020, which claims the benefit of priority of U.S. Provisional Application No. 62/794,978, filed Jan. 21, 2019, the entire contents of each of which is incorporated herein by reference.

### BACKGROUND

Concentrating therapeutic cells and transferring them from one solution into another (usually referred to as washing) are two processes involved at multiple stages of production and use of the cells. The washing and separation of materials in cellular processing is an important part of the overall efficacy of the cell therapy of choice. In particular, therapeutic cells may originally be suspended in a growth serum or in preservative materials like dimethyl sulfoxide (DMSO). Separating the cells from these fluids so the cells can be further processed is important in the overall therapeutic process of using such cellular materials. In one example, the cells are typically recovered from a bioreactor, concentrated, and transferred from culture media into an electroporation buffer prior to transduction, such as in manufacturing CAR-T cells. After expansion of cells at the final manufacturing step, they are concentrated and transferred into an appropriate solvent depending on the desired application.

Therapeutic cells are stored in specialized media to prolong the viability of these cells either through refrigeration and or freezing processes. Such specialized media may not be compatible when the therapeutic cells are introduced into a patient. It may thus be helpful to both wash and concentrate the therapeutic cells in a buffer or wash media that is biocompatible with both the therapeutic cells and with the patient. These washing and concentration processes conventionally involve the use of centrifugation and physical filtration. The washing step may be repeated a number of times. For example, the specialized media (which can be pyrogenic or otherwise harmful) may be fully removed with multiple wash steps, and the cells may be suspended in a new buffer or wash solution. During this washing process, many of the cells are degraded or destroyed through the centrifugation and physical filtration processes. Moreover, the filtration process can be rather inefficient and may entail a non-sterile intrusion into the environment for batch processing, whereby the cell culture is exposed to possible pathogens or outside cellular influences that would be harmful to the target cell culture. Further yet, with these physical filtration processes, biological waste is generated through the use of multiple physical filters which may incur additional steps for proper disposal. The cost and timeliness of this process is also not conducive to a fast or low-cost process of preparing the cells for introduction to the patient.

### BRIEF SUMMARY

The present disclosure provides methods and systems for replacing or augmenting conventional centrifugation and physical filtration processes along with the multiple washing steps with a simpler, lower cost, and more friendly process for particles such as therapeutic cells. The methods/processes can be performed in a sterile environment and in a continuous form.

Disclosed herein are methods of washing particles, which may be cells. In some example methods, an initial mixture of a first media and the particles is fed to a flow chamber of an acoustophoretic device. The first media may contain preservatives such as dimethyl sulfoxide (DMSO) which are undesirable for future applications/uses of the particles. The acoustophoretic device has at least one ultrasonic transducer that includes a piezoelectric material and is configured to be driven to create a multi-dimensional acoustic standing wave in the flow chamber. At least a portion of the particles are trapped in the multi-dimensional acoustic standing wave. A second media is flowed through the flow chamber to wash out the first media while the particles are retained in the multidimensional acoustic standing wave. The particles may thus experience a media exchange, where the first media is exchanged for the second media.

In some examples, the volume of the second media used to perform the wash process may be equivalent to a volume of the flow chamber. In some examples, the volume of the second media used to perform the wash process may be multiples of or portions of the volume of the flow chamber. The second media can be a biocompatible wash or a buffer solution.

The particles may be cells. The cells may be Chinese hamster ovary (CHO) cells, NSO hybridoma cells, baby hamster kidney (BHK) cells, human cells, regulatory T-cells, Jurkat T-cells, CAR-T cells, B cells, or NK cells, peripheral blood mononuclear cells (PBMCs), algae, plant cells, bacteria, or viruses. The cells may be attached to microcarriers.

Sometimes, the piezoelectric material of the at least one ultrasonic transducer is in the form of a piezoelectric array formed from a plurality of piezoelectric elements. Each piezoelectric element can be physically separated from surrounding piezoelectric elements by a potting material. The piezoelectric array can be present on a single crystal, with one or more channels separating the piezoelectric elements from each other. Each piezoelectric element can be individually connected to its own pair of electrodes. The piezoelectric elements can be operated in phase with each other, or operated out of phase with each other. The acoustophoretic device may further comprise a cooling unit for cooling the at least one ultrasonic transducer.

In various embodiments, the initial mixture may have a density of about 0.5 million particles/mL to about 5 million particles/mL. The concentrated volume can be 25 to about 50 times less than a volume of the initial mixture. The concentrated volume may have a particle density of 25 to about 50 times greater than a particle density of the initial mixture.

Also disclosed in various embodiments are methods of recovering greater than 90% of cells from a cell culture. An initial mixture of a first media and the cell culture is fed through a flow chamber of an acoustophoretic device, the acoustophoretic device comprising at least one ultrasonic transducer including a piezoelectric material that is configured to be driven to create a multi-dimensional acoustic standing wave in the flow chamber. The at least one ultrasonic transducer is driven to create a multi-dimensional acoustic standing wave in the flow chamber, and thus to concentrate the cell culture within the acoustic standing wave. The initial mixture has an initial cell density of about 0.5 million cells/mL to about 5 million cells/m L, and the concentrated cell culture has a cell density at least 25 times greater than the initial cell density.

In some embodiments, the concentrated cell culture has a cell density of 25 to about 50 times greater than the initial cell density. In other embodiments, a volume of the concentrated cell culture is 25 to about 50 times less than a volume of the initial mixture. The concentrated cell culture can be obtained in about 35 minutes or less.

Also disclosed are acoustophoretic devices, comprising: a flow chamber having a fluid inlet, a first outlet, and a second outlet; at least one ultrasonic transducer proximate a first wall of the flow chamber, at least one ultrasonic transducer including a piezoelectric material that is adapted to be driven to create a multi-dimensional acoustic standing wave; a reflector on a second wall of the flow chamber opposite the at least one ultrasonic transducer; and a thermoelectric generator located between the at least one ultrasonic transducer and the first wall of the flow chamber.

The acoustophoretic device may have a concentrated volume of about 25 mL to about 75 mL. The acoustophoretic device may have a cell capacity of about 4 billion to about 40 billion cells. Various lines can connect the acoustophoretic device to containers that provide or receive various materials to/from the acoustophoretic device.

These and other non-limiting characteristics are more particularly described below.

### BRIEF DESCRIPTION OF THE DRAWINGS

The following is a brief description of the drawings, which are presented for the purposes of illustrating the example embodiments disclosed herein and not for the purposes of limiting the same.

FIG. 1 illustrates an example acoustophoresis process using a transducer and reflector to create an acoustic standing wave for trapping particles and separating them from a fluid by enhanced gravitational settling.

FIG. 2 illustrates an example cell concentration and washing process (“diafiltration”) according to the present disclosure using acoustophoresis.

FIG. 3 illustrates another example cell concentration and washing process (push through) according to the present disclosure using acoustophoresis.

FIG. 4 shows six photographs that, from left to right and top to bottom, show the progression of cells being trapped in an acoustophoretic device before a second media mixture (dyed blue) is flowed into the device and gradually replaces the first media (dyed red).

FIG. 5 is a graph showing the performance of an acoustophoretic device. The x-axis is elapsed time (minutes) and runs from 0 to 40 in increments of 5. The left-side y-axis is permeate density reduction (%) and runs from 0 to 100 in increments of 10. The right-side y-axis is permeate cell density (×10⁶ cells/mL) and runs from 0.00 to 2.00 in increments of 0.20. The uppermost solid line represents permeate reduction density (%). The lowermost solid line represents permeate cell density. The middle line running substantially horizontally across the page represents feed cell density for reference purposes.

FIG. 6 is a graph showing the T-cell concentration performance of an acoustophoretic process according to the present disclosure with a low cell density culture. The x-axis is elapsed time (minutes) and runs from 0 to 25 in increments of 5. The left-side y-axis is percent reduction (%) and runs from 0 to 100 in increments of 10. The right-side y-axis is cell density (×10⁶ cells/mL) and runs from 0.00 to 1.60 in increments of 0.20. The upper solid line represents permeate reduction (%). The lower solid line represents permeate cell density. The dashed line represents feed cell density for reference purposes.

FIG. 7 is a graph showing the percent density reduction (PDR) dependency on concentration and flow rate for an acoustophoretic process according to the present disclosure. The x-axis is time (minutes) and runs from 0 to 40 in increments of 5. The y-axis is permeate density reduction (%) and runs from 0 to 100 in increments of 10. The line having circle-shaped data points represents a mixture having an initial cell concentration of 5×10⁶ cells/mL. The line having x-shaped data points represents a mixture having an initial cell concentration of 3×10⁶ cells/mL. The line having triangle-shaped data points represents a mixture having an initial cell concentration of 1×10⁶ cells/mL at a flow rate of 20 mL/minute. The line having diamond-shaped data points represents a mixture having an initial cell concentration of 1×10⁶ cells/mL at a flow rate of 10 mL/minute.

FIG. 8 is a graph showing the T-cell performance for an acoustophoretic process according to the present disclosure with a high cell density culture. The x-axis is elapsed time (minutes) and runs from 0 to 25 in increments of 5. The left-side y-axis is percent reduction (%) and runs from 0 to 100 in increments of 10. The right-side y-axis is cell density (×10⁶ cells/mL) and runs from 0.00 to 3.00 in increments of 0.50. The upper solid line represents permeate density reduction (%). The lower solid line represents permeate cell density. The dashed line represents feed cell density for reference purposes.

FIG. 9A is a perspective view of an example acoustophoretic device according to the present disclosure including a cooling unit for cooling the transducer. FIG. 9B is an exploded view of the device of FIG. 9A.

FIG. 10 is a graph showing the temperature profile of an acoustophoretic device without active cooling. The x-axis is elapsed time (minutes) and runs from 0.00 to 20.00 in increments of 2.00. The y-axis is temperature (° C.) and runs from 17.00 to 33.00 in increments of 2.00. The lowermost line along the right side of the graph represents the feed temperature (° C.). The uppermost line along the right side of the graph represents the core temperature (° C.). The middle line along the right side of the graph represents the permeate temperature (° C.).

FIG. 11 is a graph showing the temperature profile of an acoustophoretic device with active cooling of the transducer. The x-axis is elapsed time (minutes) and runs from 0.00 to 20.00 in increments of 2.00. The y-axis is temperature (° C.) and runs from 17.00 to 33.00 in increments of 2.00. The lowermost line along the right side of the graph represents the feed temperature (° C.). The uppermost line along the right side of the graph represents the core temperature (° C.). The middle line along the right side of the graph represents the permeate temperature (° C.).

FIGS. 12A and 12B illustrate a process for concentrating, washing, and/or separating microcarriers and cells according to the present disclosure. The top portion of FIG. 12A represents a first step of receiving complexes of microcarriers and cells surrounded by a bioreactor serum from a bioreactor and concentrating the microcarrier/cell complexes in an acoustophoretic device according to the present disclosure. The lower portion of FIG. 12A represents a second step of washing the concentrated microcarriers with attached cells to remove the bioreactor serum. The top portion of FIG. 12B represents a third step of trypsinizing, or disassociating, the microcarriers and cells and a fourth step of separating the microcarriers from the cells. The bottom portion of FIG. 12B represents a final wash and concentrate step that can be employed as desired.

FIG. 13 shows the concentration of microcarriers with attached T cells in the feed into the acoustophoretic device (top row of photographs) and the concentration of separated microcarriers and T cells in the permeate drawn out of the acoustophoretic device (bottom row of photographs). The dark circular items indicate microcarriers, and the lighter area indicates T cells.

FIG. 14 shows microscopic images of the concentration of microcarriers with attached T cells in the feed and the concentration of separated microcarriers and T cells in the permeate.

FIG. 15 is a schematic of an example acoustophoretic system according to the present disclosure showing the flow path of the feed material through the system.

FIG. 16 is a schematic of the example acoustophoretic system of FIG. 15 showing the flow path of the wash material through the system.

FIG. 17 is a schematic of the example acoustophoretic system of FIG. 15 showing draining of the system.

FIG. 18 is a two-axis graph showing the results of trial A. The left-hand y-axis is the percent reduction of cells in the permeate, and runs from 0 to 100% at intervals of 20%. The right-hand y-axis is the cell density of the permeate in units of million cells/mL, and runs from 0 to 1.00 at intervals of 0.20. The x-axis is elapsed time in minutes, and runs from 0 to 33 minutes at intervals of 3. The dotted line indicates the initial cell density, which was 0.98 million cells/mL.

FIG. 19 is a two-axis graph showing the results of trial B. The left-hand y-axis is the percent reduction of cells in the permeate, and runs from 0 to 100% at intervals of 20%. The right-hand y-axis is the cell density of the permeate in units of million cells/mL, and runs from 0 to 1.00 at intervals of 0.20. The x-axis is elapsed time in minutes, and runs from 0 to 33 minutes at intervals of 3. The dotted line indicates the initial cell density, which was 0.85 million cells/mL.

FIG. 20 is a two-axis graph showing the results of trial C. The left-hand y-axis is the percent reduction of cells, and runs from 0 to 100% at intervals of 20%. The right-hand y-axis is the cell density in units of million cells/mL, and runs from 0 to 4.00 at intervals of 1.00. The x-axis is elapsed time in minutes, and runs from 0 to 30 minutes at intervals of 3. The dotted line indicates the initial cell density, which was 4.08 million cells/mL.

FIG. 21 is a graph showing viability of cells in the concentrate wash process.

FIG. 22 is a graph showing cell density versus power.

FIG. 23 is a side cross sectional diagram of a concentrate wash device for low cell density applications with a faceted reflector and a concentrate wash device for high cell density applications with a planar reflector.

FIG. 24 is a side cross sectional diagram of the devices in FIG. 23 showing operation of the devices with low and high cell density applications.

FIG. 25 is a series of photographs showing processing of T-cells with the concentrate wash device.

FIG. 26 is a graph showing waste viable cell density over time.

FIG. 27 is a diagram of an acoustic concentrate-wash system.

FIG. 28 is a two-axis bubble graph of viable cell recovery versus billions of cells processed. The y-axis is percentage of viable cell recovery ranging from 70% to 100%. The x-axis is billions of cells processed ranging from 0 to 30. The legend indicates initial wash buffer, represented on the bubble graph as ranging from about 2.5 billion to about 25 billion on the x-axis. The legend also indicates Optimized wash buffer, represented on the bubble graph as ranging from about 1.25 billion to about 2.5 billion on the x-axis.

FIGS. 29A, 29B, 29C, 29D and 29E are a table of parameters for viable cell densities. The rows represent volume in mL, and the columns are viable cell densities in millions of cells per mL. FIG. 29E provides a legend for the table, indicating operating parameters for the different regions with different conditions.

FIG. 30 is a graph showing concentration versus wash volumes. The y-axis is a concentration ratio of current concentration to original concentration and ranges from −0.2 to 1.2. The x-axis is the number of wash volumes and ranges from 0.0 to 6.0.

FIG. 31 is a diagram of an acoustic aggregate separation system for human pluripotent stem cells (hPSC).

FIG. 32 is a graph of retention of hPSC aggregates versus power. The y-axis is percentage of aggregate retention and ranges from 75% to 100%. The x-axis is Power in watts and ranges from 0 to 35.

FIG. 33 is a bar graph of bioreactor aggregate and single cell separation. The y-axis is the percentage of fractionation between aggregates and single cells and ranges from 0% to 100%. The x-axis is the number of cycles and ranges from an initial amount to a 2^{nd} cycle.

FIG. 34 is a sample dot graph showing T-cell expansion and viability, and a bar graph showing a sample T-cell viability and T-cell type composition before and after acoustic concentrate-wash processing. The dot graph left y-axis is the fold number of cell expansions and ranges from 0 to 35. The dot graph right y-axis is viability percentage and ranges from 50% to 100%. The dot graph x axis is process time in days and ranges from 0 to 12. The bar graph y-axis is the percentage of cells and ranges from 0% to 100%. The x-axis components are numbers of viable cells, total T-cells, CD4+ T-cells, and CD8+ T-cells, before and after acoustic concentrate-wash processing.

FIG. 35 is a series of flow cytometry graphs showing initial and concentrated result for CD4 and CD8 T-cells.

FIG. 36 is a diagram of an end-to-end process for manufacturing a cell therapy product.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. Furthermore, it should be understood that the drawings are not to scale.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named components/steps and permit the presence of other components/steps. However, such description should be construed as also describing compositions or processes as “consisting of” and “consisting essentially of” the enumerated components/steps, which allows the presence of only the named components/steps, along with any impurities that might result therefrom, and excludes other components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

A value modified by a term or terms, such as “about” and “substantially,” may not be limited to the precise value specified. The approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, e.g. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, e.g. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, e.g. ground level. The terms “upwards” and “downwards” are also relative to an absolute reference; an upwards flow is always against the gravity of the earth.

The present application refers to “the same order of magnitude.” Two numbers are of the same order of magnitude if the quotient of the larger number divided by the smaller number is a value of at least 1 and less than 10.

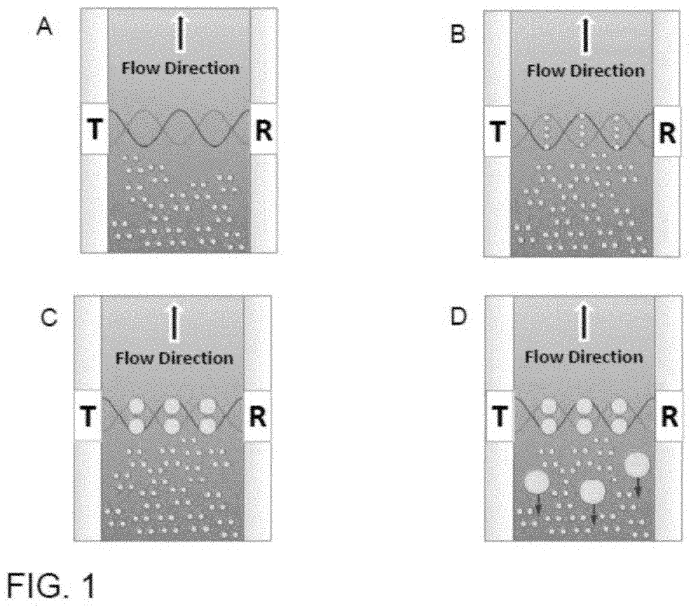

The acoustophoretic technology of the present disclosure employs acoustic standing waves to concentrate, wash, and/or separate materials (such as particles or a secondary fluid) in a primary or host fluid. In particular, as shown in the upper left image (A) of FIG. 1, an ultrasonic transducer T creates an acoustic wave in the fluid, which interacts with a reflector R positioned across from the ultrasonic transducer to create an acoustic standing wave. Although a reflector R is illustrated in FIG. 1, another transducer may be used to reflect and/or generate acoustic energy to form the acoustic standing wave.

As shown in the upper right image (B) of FIG. 1, as the host fluid and material entrained in the host fluid flows upwards through the acoustic standing wave, the acoustic standing wave(s) traps (retains or holds) the material (e.g., secondary phase materials, including fluids and/or particles). The scattering of the acoustic field off the material results in a three-dimensional acoustic radiation force, which acts as a three-dimensional trapping field.

The three-dimensional acoustic radiation force generated in conjunction with an ultrasonic standing wave is referred to in the present disclosure as a three-dimensional or multi-dimensional standing wave. The acoustic radiation force is proportional to the particle volume (e.g. the cube of the radius) of the material when the particle is small relative to the wavelength. The acoustic radiation force is proportional to frequency and the acoustic contrast factor. The acoustic radiation force scales with acoustic energy (e.g. the square of the acoustic pressure amplitude). For harmonic excitation, the sinusoidal spatial variation of the force drives the particles to the stable positions within the standing waves. When the acoustic radiation force exerted on the particles is stronger than the combined effect of fluid drag force and buoyancy and gravitational force, the particle can be trapped within the acoustic standing wave field, as shown in the upper right image (B) of FIG. 1.

As can be seen in the lower left image (C) of FIG. 1, this trapping results in coalescing, clumping, aggregating, agglomerating, and/or clustering of the trapped particles. Additionally, secondary inter-particle forces, such as Bjerkness forces, aid in particle agglomeration.

As the particles continue to coalesce, clump, aggregate, agglomerate, and/or cluster the particles can grow to a certain size at which gravitational forces on the particle cluster overcome the acoustic radiation force. At such size, the particle cluster can fall out of the acoustic standing wave, as shown in the lower right image (D) of FIG. 1.

Desirably, the ultrasonic transducer(s) generate a three-dimensional or multi-dimensional acoustic standing wave in the fluid that exerts a lateral force on the suspended particles to accompany the axial force so as to increase the particle trapping capabilities of the standing wave. A planar or one-dimensional acoustic standing wave may provide acoustic forces in the axial or wave propagation direction. The lateral force in planar or one-dimensional acoustic wave generation may be two orders of magnitude smaller than the axial force. The multi-dimensional acoustic standing wave may provide a lateral force that is significantly greater than that of the planar acoustic standing wave. For example, the lateral force may be of the same order of magnitude as the axial force in the multi-dimensional acoustic standing wave.

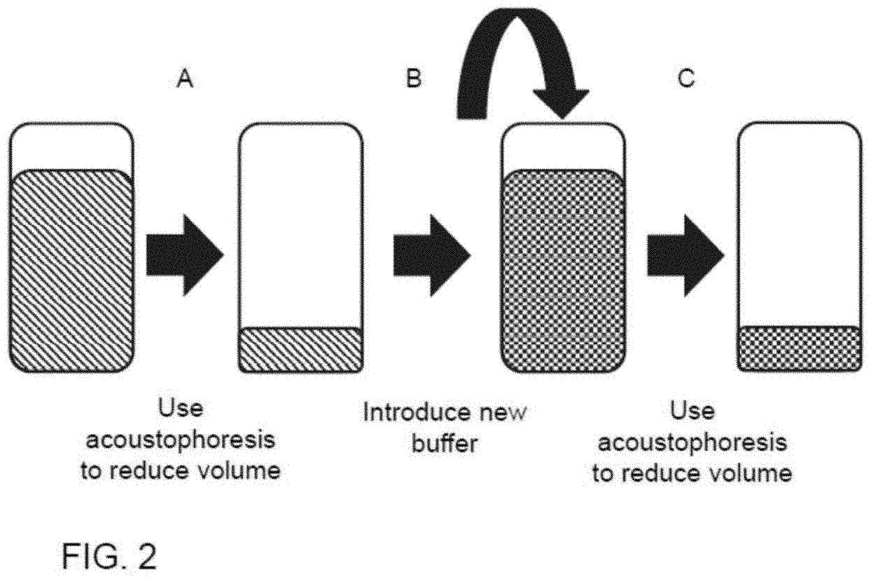

The acoustic standing waves of the present disclosure can be used to trap particles (e.g. therapeutic cells such as T cells, B cells, NK cells) suspended in a first media in the standing wave. The first media can then be replaced with a second media (e.g., a biocompatible wash or buffer solution). Put another way, the host fluid of the particles can be replaced. Prior to replacing the first media with the second media, acoustophoresis can be used to perform a diafiltration process, as shown in FIG. 2.

In FIG. 2, starting with an initial mixture that has a low cell density of, for example, less than 1×10⁶ cells/mL, acoustophoresis can be used to reduce the volume of the initial mixture, for example by at least 10×, including 20× and up to 100× or more. The cell concentration may be increased by at least 10×, including 20× and up to 100× or more. This initial reduction process is the first volume reduction step (A). Next, the second media (e.g., a biocompatible wash or buffer solution) can be introduced to at least partially displace the first media, as indicated in step (B). Next, the new mixture of the cells and second media can be subjected to an acoustophoretic volume reduction step (C). This series of operations is referred to as a “diafiltration” process.

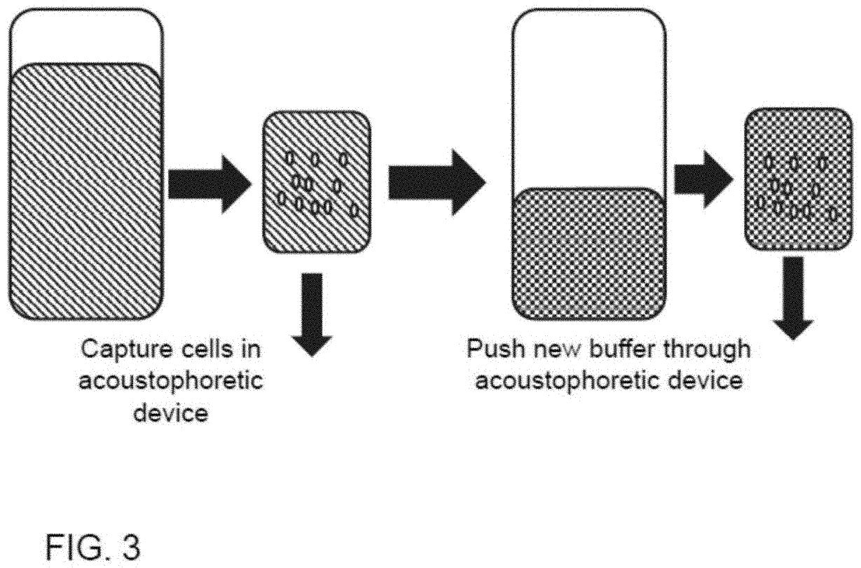

FIG. 3 illustrates a single-step, push-through process in which particles/cells are trapped in the acoustic standing wave and held in the acoustophoretic device. The second media (e.g., a biocompatible wash or buffer solution) is then flowed into the acoustophoretic device to effectively “wash out” the first media. With the push-through process, more than 90%, including up to 99% or more, of the first media can be removed from the particles/cells. The push-through process can be employed as a continuous, single-use process that uses less buffer solution and less time than the diafiltration process of FIG. 2. Feed volumes for the process can be from 500 mL to 3 L, processing time can be less than 60 minutes, the incoming feed density can be from less than about one million cells per mL (1 M/mL) to about forty million cells per mL (40 M/mL). The process has no effect on viability for the cells, and the final concentrate volume is less than 7 mL with 1 M/mL and less than 50 mL with 40 M/mL. The concentration factor, from a beginning concentration with 1 M/mL is 15 times, e.g., 105 mL to 7 mL, and from a beginning concentration with 40 M/mL is 140 times, e.g., 7 L to 50 mL.

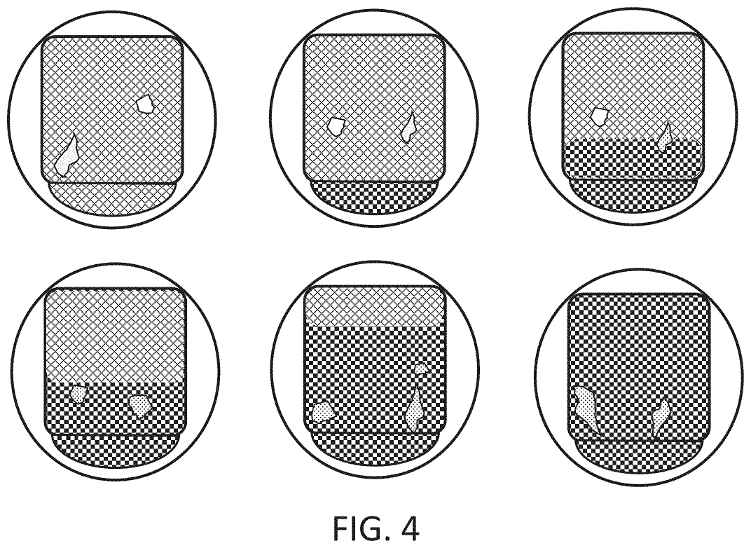

FIG. 4 shows six photographs that, from left to right and top to bottom, show the progression of cells being trapped in an acoustophoretic device before a second media mixture (dyed blue) is flowed into the device and gradually replaces the first media (dyed red). In FIG. 4, a 150 mL feed volume was used with 80 mL of electroporation media wash for the second media. The concentrate was drawn off at a flow rate of 10 mL/minute. As can be seen in these pictures, over time the first media is replaced with the second media.

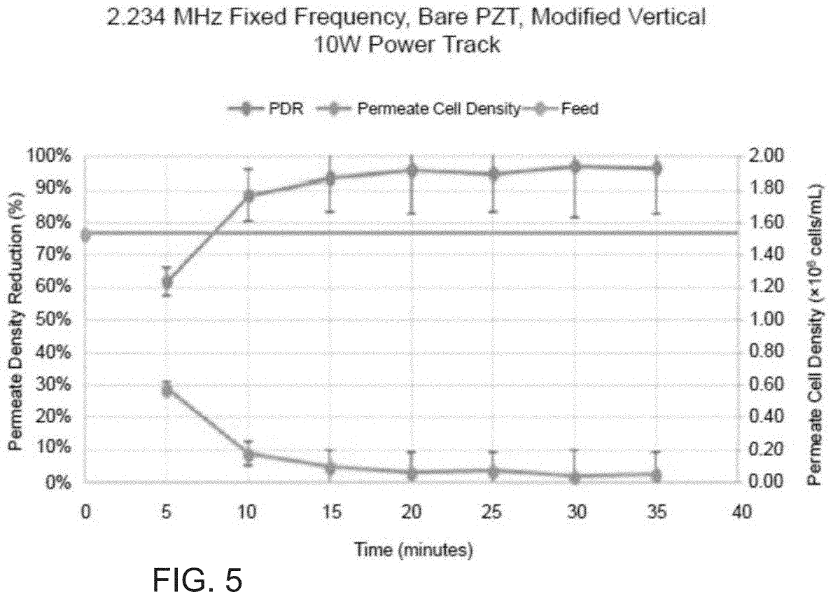

FIG. 5 is a graph showing the performance of an acoustophoretic device operated at a fixed frequency of 2.234 MHz for a mixture having a feed cell density of about 1.5×10⁶ cells/mL. As can be seen, the device achieved a permeate density reduction (PDR) of greater than 95% over about 35 minutes and a permeate cell density of less than 0.10×10⁶ cells/mL over the same time period.

The piezoelectric transducer(s) of the acoustophoretic devices and systems of the present disclosure can be single monolithic piezoelectric materials or can be made from an array of piezoelectric materials. The piezoelectric material can be a ceramic material, a crystal or a polycrystal, such as PZT-8 (lead zirconate titanate).

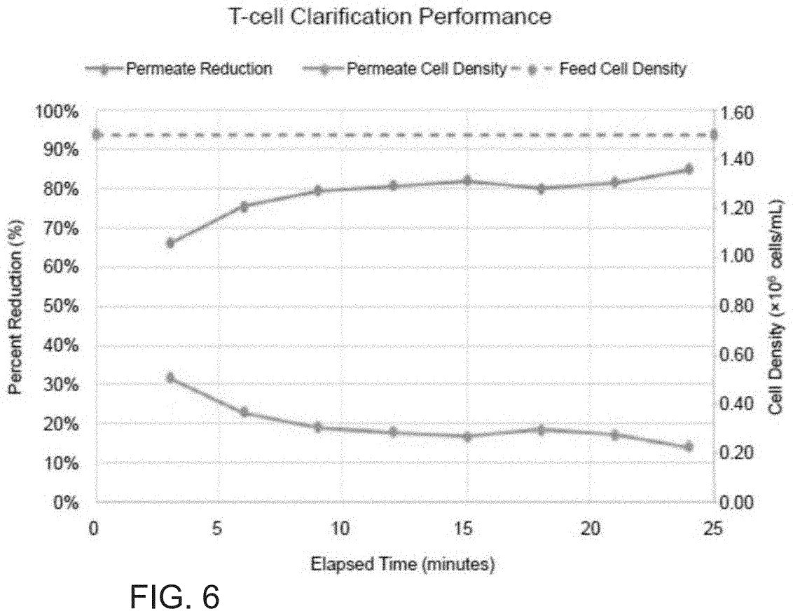

The concentration efficiency of the acoustophoretic device was tested. First, a T-cell suspension having a cell density of 1×10⁶ cells/mL was used. A feed volume of between about 500 and 1000 mL was used at a flow rate of 10-15 mL/minute. The results are graphically depicted in FIG. 6. The device exhibited a concentration factor of between 10× and 20×, a 90% cell recovery, and a 77% washout efficiency (i.e., the amount of the first media that was displaced by the second media) over ten minutes of testing. A 10° C. temperature increase was observed.

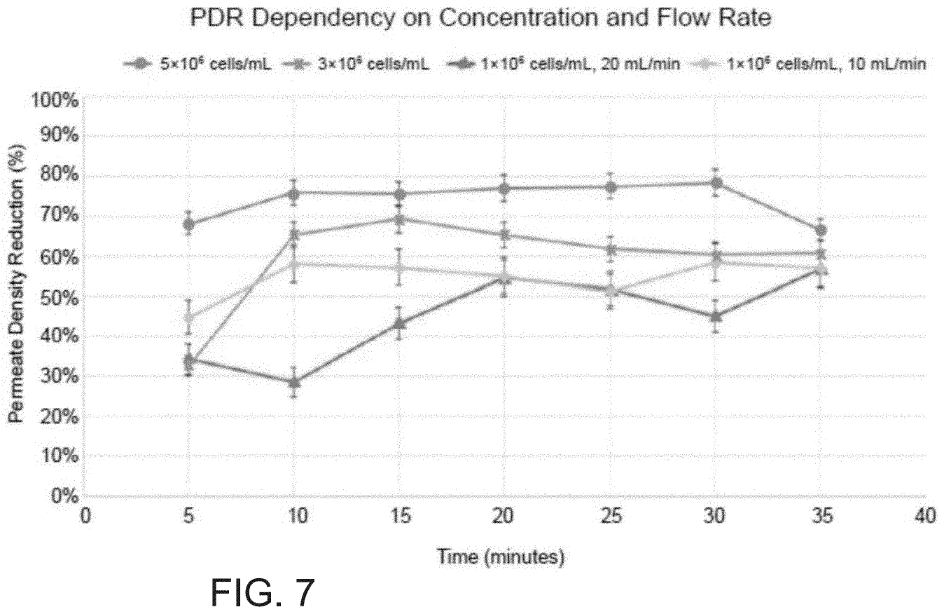

A yeast mixture was then used to test the dependency of the percent density reduction (PDR) on concentration and flow rate. The results are graphically depicted in FIG. 7. As seen here, the higher initial cell concentrations generally resulted in a greater PDR. Additionally, the varied flow rate (from 20 mL/min to 10 mL/min) did not have an observed effect on the PDR.

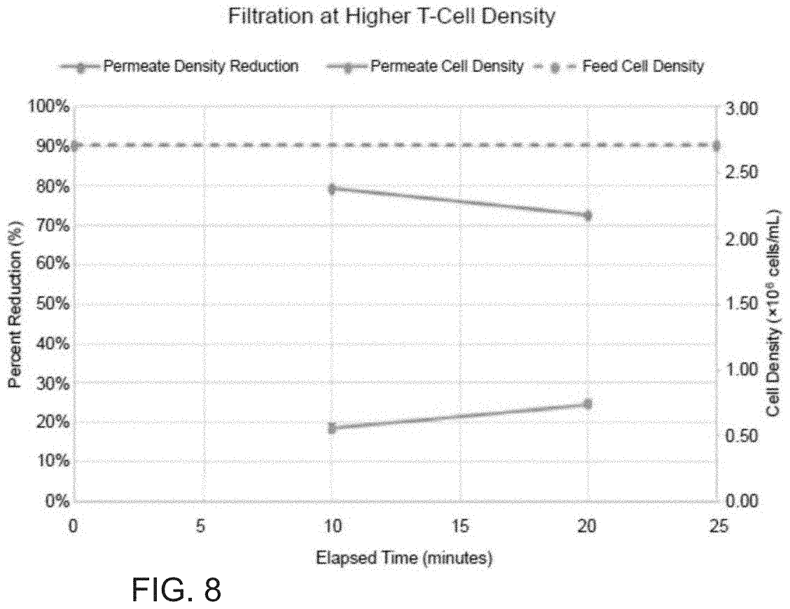

The concentration efficiency of the acoustophoretic device was again tested with a higher cell density. A T-cell suspension having a cell density of 5×106 cells/mL was used. A feed volume of 1000 mL was used at a flow rate of 10-15 mL/minute. The results are graphically depicted in FIG. 8. The device exhibited a concentration factor of better than 10×, a 90% cell recovery, and a 77% washout efficiency over one hour of testing. A 10° C. temperature increase was again observed.

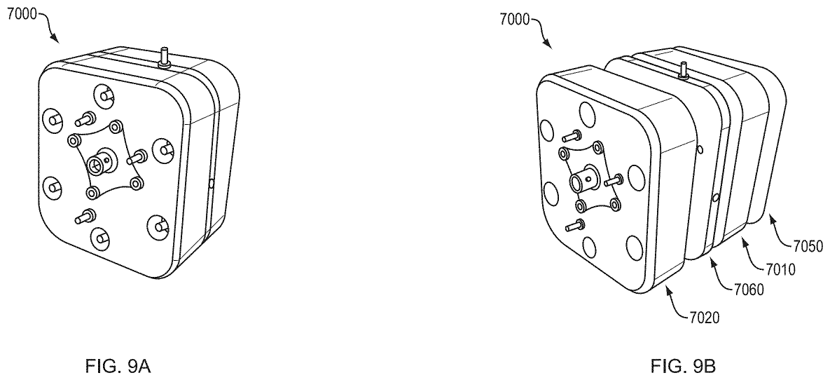

During testing, it was also discovered that active cooling of the ultrasonic transducer led to greater throughput and efficiency and more power. As such, a cooling unit was developed for actively cooling the transducer. FIG. 9A illustrates an acoustophoretic device 7000 containing a cooling unit, in a fully assembled condition. FIG. 9B illustrates the device 7000, with the various components in a partially exploded view. Referring now to FIG. 9B, the device includes an ultrasonic transducer 7020 and a reflector 7050 on opposite walls of a flow chamber 7010. It is noted that the reflector 7050 may be made of a transparent material, such that the interior of the flow chamber 7010 can be seen. The ultrasonic transducer is proximate a first wall of the flow chamber. The reflector is proximate a second wall of the flow chamber or can make up the second wall of the flow chamber. A cooling unit 7060 is located between the ultrasonic transducer 7020 and the flow chamber 7010. The cooling unit 7060 is thermally coupled to the ultrasonic transducer 7020. In this figure, the cooling unit is in the form of a thermoelectric generator, which converts heat flux (i.e. temperature differences) into electrical energy using the Seebeck effect, thus removing heat from the flow chamber. Put another way, electricity can be generated from undesired waste heat while operating the acoustophoretic device.

It is noted that the various inlets and outlets (e.g. fluid inlet, concentrate outlet, permeate outlet, recirculation outlet, bleed/harvest outlet) of the flow chamber are not shown here. The cooling unit can be used to cool the ultrasonic transducer, which can be particularly advantageous when the device is to be run continuously with repeated processing and recirculation for an extended period of time (e.g., perfusion).

Alternatively, the cooling unit can also be used to cool the fluid running through the flow chamber 7010. For desired applications, the cell culture should be maintained around room temperature (˜20° C.), and at most around 28° C. This is because when cells experience higher temperatures, their metabolic rates increase. Without a cooling unit, however, the temperature of the cell culture can rise as high as 34° C.

These components are modular and can be changed or switched out separately from each other. Thus, when new revisions or modifications are made to a given component, the component can be replaced while the remainder of the system stays the same.

The goal is to begin with a culture bag having a volume of about 1 liter (L) to about 2 L, with a density of about 1 million cells/mL, and concentrate this bag to a volume of about 25 mL to about 30 mL, and then to wash the growth media or exchange the media within a time of about one hour (or less). Desirably, the system can be made of materials that are stable when irradiated with gamma radiation.

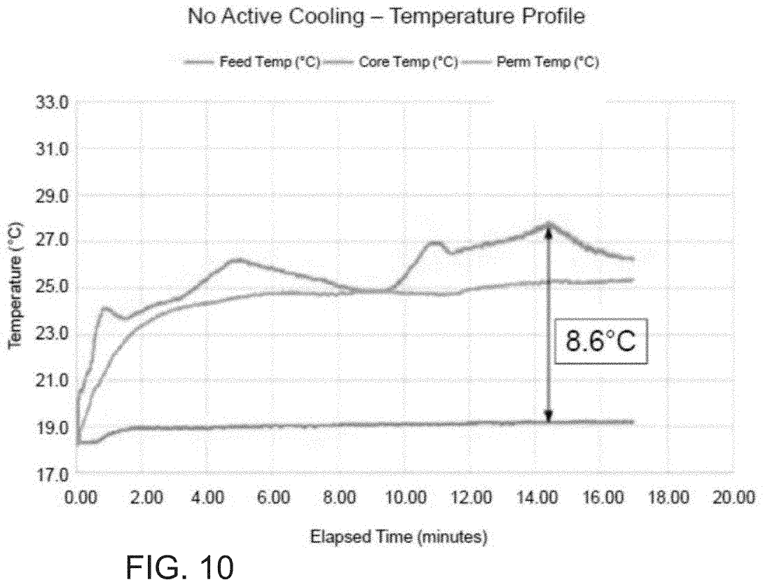

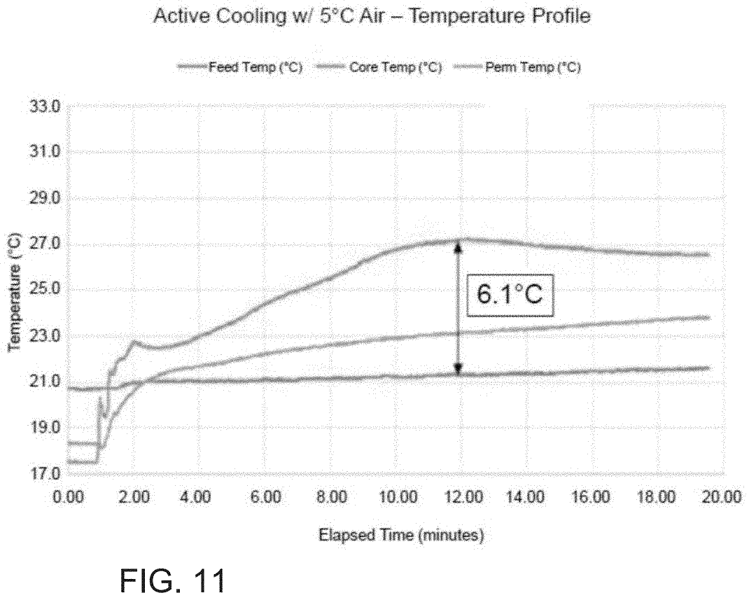

The advantages of providing a cooling unit for the transducer can be seen in FIG. 10 and FIG. 11. FIG. 10 graphically shows the temperature profile of the acoustophoretic device without any active cooling (e.g., without a cooling unit for the transducer). As seen in FIG. 10, the temperature difference between the feed and the core (e.g., the transducer) was 8.6° C. FIG. 11 graphically shows the temperature profile of the acoustophoretic device with active cooling (e.g., with a cooling unit for the transducer). As seen in FIG. 11, through the use of active cooling the temperature difference between the feed and the core was reduced to 6.1° C.

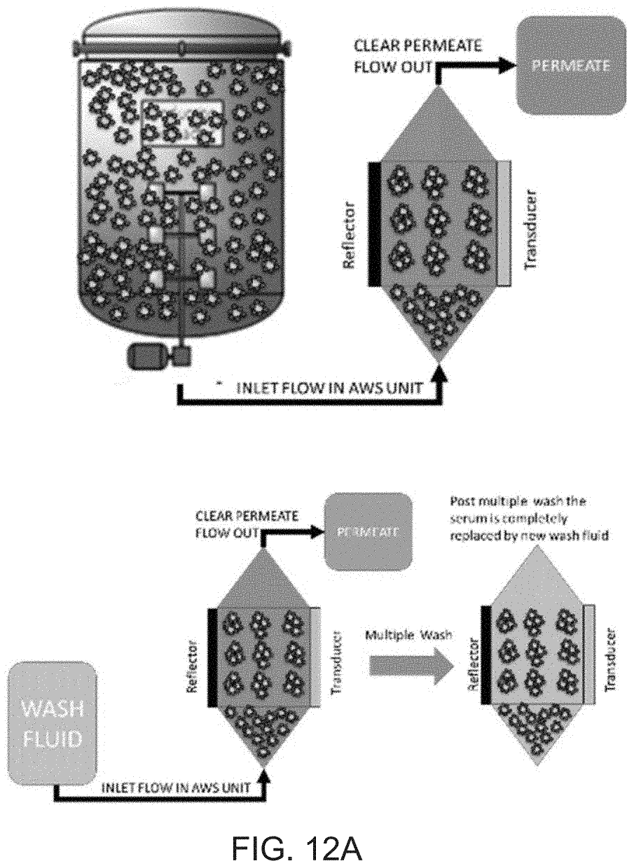

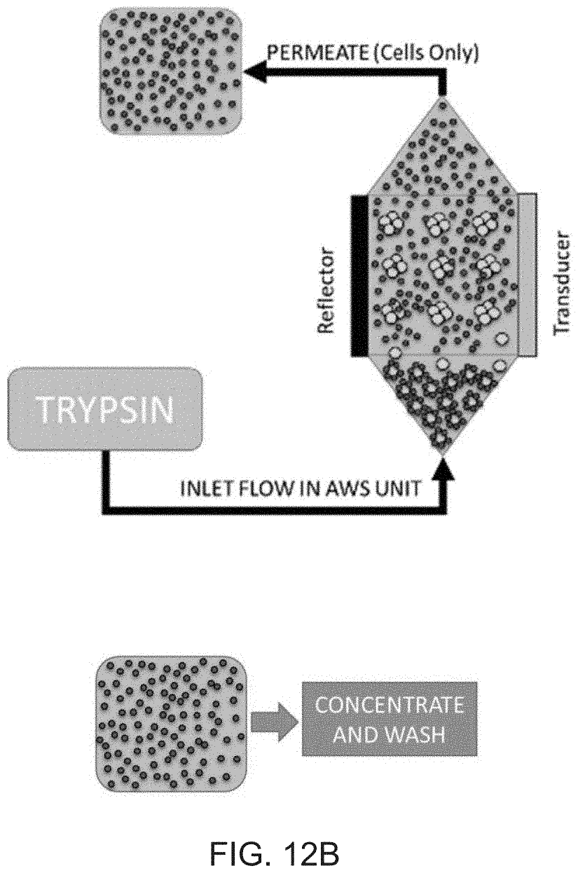

FIGS. 12A and 12B illustrate a four-step process (with an optional fifth step) for concentrating, washing, and separating microcarriers from cells. The first step in the process involves concentrating the microcarriers with attached cells in an acoustophoretic device, such as those described herein. The microcarriers and attached cells can be introduced to the acoustophoretic device by receiving the microcarriers with attached cells from a bioreactor. In the bioreactor, the microcarriers and cells are suspended in a first media (e.g., growth serum or preservative material used to keep the cells viable in the bioreactor). The microcarriers with attached cells surrounded by the first media are concentrated by the acoustic standing wave(s) generated in the acoustophoretic device. In a second step, the concentrated microcarriers with attached cells are then washed with a second media to remove the first media (e.g., bioreactor growth serum or preservative material). The third step is to then introduce a third media containing an enzyme into the acoustophoretic device to detach the cells from the microcarriers through enzymatic action of the second media. In particular embodiments, trypsin is the enzyme used to enzymatically detach the cells from the microcarriers. The multi-dimensional acoustic standing wave can then be used to separate the cells from the microcarriers. Usually, this separation is done by trapping the microcarriers in the multi-dimensional acoustic standing wave, while the detached cells pass through with the third media. However, the cells can be trapped instead, if desired. Finally, the separated cells may optionally be concentrated and washed again, as desired.

After being concentrated and trapped/held in the multi-dimensional acoustic standing wave, the microcarriers can coalesce, clump, aggregate, agglomerate, and/or cluster to a critical size at which point the microcarriers fall out of the acoustic standing wave due to enhanced gravitational settling. The microcarriers can fall into a collector of the acoustophoretic device located below the acoustic standing wave, to be removed from the flow chamber.

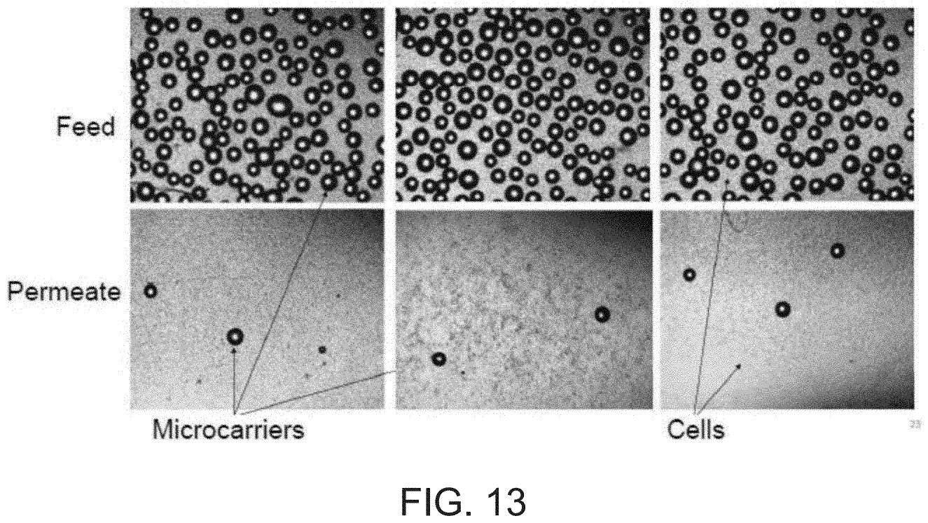

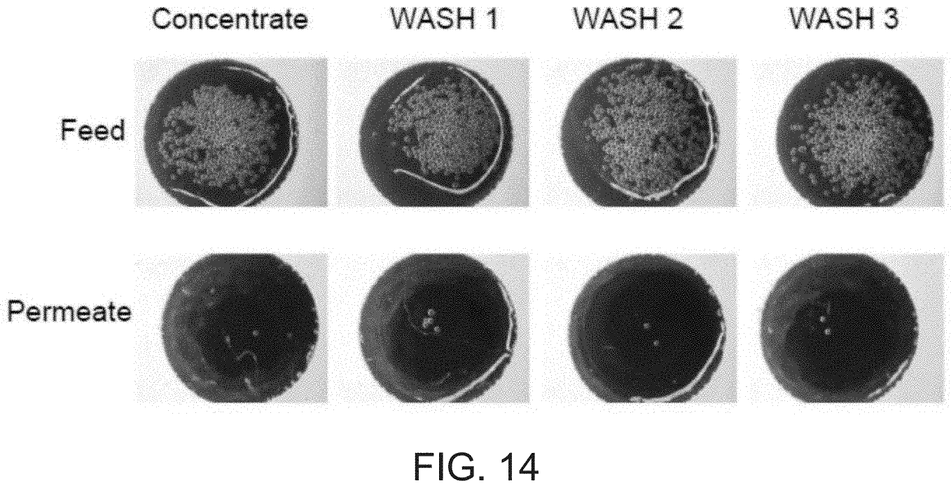

FIG. 13 shows the presence of SoloHill microcarriers and T-Cells in the acoustophoretic device under 4× magnification. The top row of images show the microcarriers and cells in the feed before acoustophoresis. The bottom row of images show the microcarriers and cells in the permeate after the cells have been separated out by acoustophoresis. The difference in the number of microcarriers with the application of acoustophoresis evidences the feasibility of using the device for trapping the microcarriers in the device and separating the cells therefrom. The feasibility of this technique and the results are further evidenced by the images in FIG. 14, which show microscopic images of the microcarriers and cells in the feed (top row of images) and permeate (bottom row of images) after concentration and the first, second, and third washes, from left to right.

The testing of the acoustophoretic concentrating, washing, and separating process showed that the process is appropriate for cell therapy and microcarrier applications. The concentrate and wash steps were performed with a resulting efficiency of greater than 99%, and the separating step e.g., separating the cells from the microcarriers, was performed with greater than 98% efficiency.

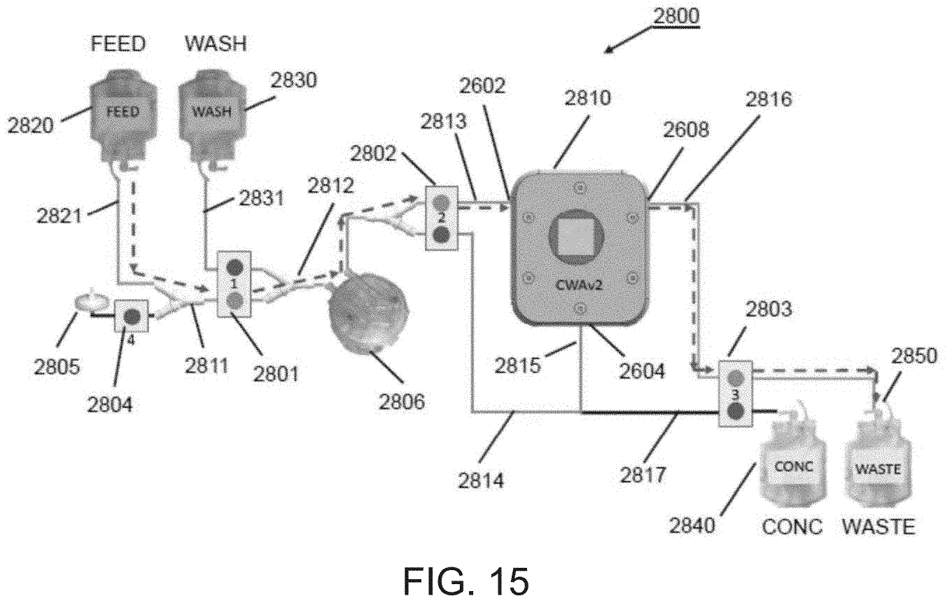

FIGS. 15-17 illustrate another example embodiment of an acoustophoretic system/process 2800 including a disposable acoustophoretic device 2810 with solenoid pinch valves that control the flow of fluid therethrough. Starting from the left-hand side of FIG. 15, the system includes a feed tank 2820, a wash tank 2830, and an air intake 2805. The air intake 2805 runs through an air intake valve 2804. Feed line 2821 runs from the feed tank 2820. The air intake and the feed line 2821 are joined together by a Y-connector into common feed line 2811, which runs into feed selector valve 2801. A wash line 2831 runs from the wash tank 2830, and also runs into feed selector valve 2801. Feed selector valve 2801 permits only one line to be open at a given time (valves 2802, 2803 also operate in this manner). Wash line 2831 and feed line 2811 are joined together by a Y-connector downstream of the feed selector valve 2801 into input line 2812. Input line 2812 passes through pump 2806 to inflow selector valve 2802, which is downstream of the feed selector valve 2801 and upstream of the acoustophoretic device 2810. The inflow selector valve 2802 selectively controls the inflow of feed or wash into the acoustophoretic device 2810 through either feed port 2602 or wash/drain port 2604. A feed line 2813 runs from the inflow selector valve 2802 to feed port 2602. A wash line 2814 runs from the inflow selector valve 2802 to common line 2815 and into wash/drain port 2604.

On the right-hand side of FIG. 15, an outflow selector valve 2803 is located downstream of the acoustophoretic device 2810 and controls the outflow of fluid therefrom. A waste line 2816 runs from waste port 2608 through outflow selector valve 2803 and subsequently to waste tank 2850. The common line 2815 runs into drain line 2817, which then passes through outflow selector valve 2803 and subsequently to concentrate tank 2840. These tanks 2840, 2850 can be, for example, collection bags. The outflow selector 2803 thereby selectively controls the flow of fluid to the concentrate tank and waste tank.

The use of collection bags at the ends of the concentrate and waste lines advantageously creates an enclosed primary environment within which concentration, washing, and/or separation of cells and cellular materials can occur, which helps to prevent the cells/cell culture/cellular material from being exposed to possible intrusions, pathogens, or outside cellular influences that would be harmful.

FIG. 15 also illustrates the flow path of the feed material through the system. In this example embodiment, feed selector valve 2801 is operated with the bottom open (and top closed), so that the feed from feed tank 2820 flows through. Inflow selector valve 2802 is operated with the top open (and bottom closed), so that the feed material enters the acoustophoretic device 2810 via feed port 2602. The outflow selector valve 2803 is also operated with the top open (and bottom closed) so that the fluid/first media of the feed material flows through to waste tank 2850. The targeted particles in the feed material (e.g., microcarriers or cells) are trapped in the acoustophoretic device 2810 by action of a multi-dimensional acoustic standing wave(s), as explained in detail herein.

The system illustrated in FIG. 15 has an acoustic element composed of polycarbonate and stainless steel. The tubing is ⅛″ PVC thin-wall tubing that permits sterile weld feed bags to be used for cell processing. A single-use pulseless pump head NaoStedi 2×2.5 mL is used. The tubing, acoustic element and pump are double-bagged and gamma irradiated. The system permits processes including priming, recirculation, concentration, media exchange, washing and/or collection. The example system can work with feeds of up to 3 L, with a total cell capacity of about 4-billion cells and a final concentrated volume of from about 6 mL to about 50 mL, although the system can have larger or smaller parameter ranges in other example implementations.

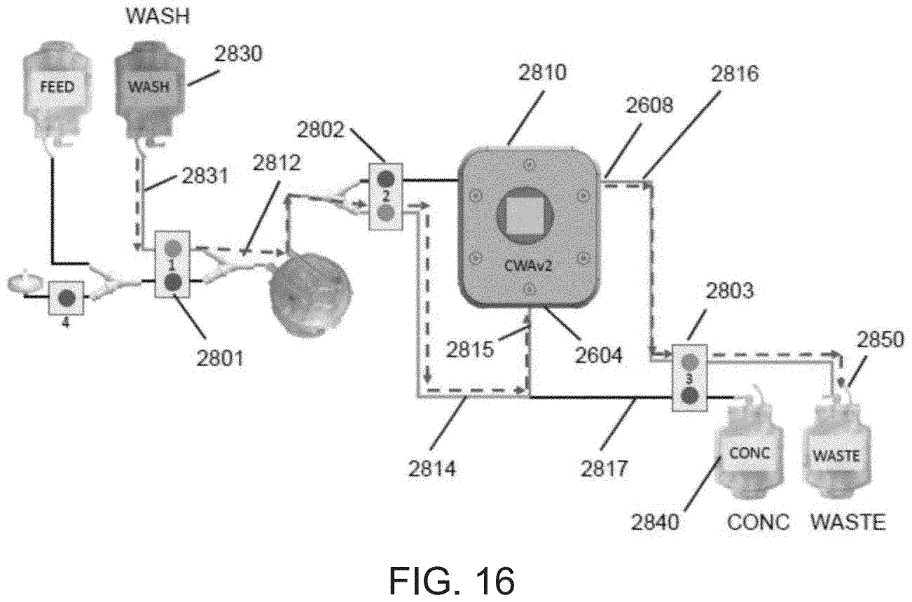

FIG. 16 illustrates the flow path of the wash material through the system. Feed selector valve 2801 is operated with the top open (and bottom closed), so that the wash material from wash tank 2830 flows through. The inflow selector valve 2802 is operated with the bottom open (and top closed) and the outflow selector valve 2803 is operated with the top open (and bottom closed). As a result, the wash material enters the acoustophoretic device 2810 via wash/drain port 2604, which operates as a wash inlet. Note that the closed outflow selector valve 2803 prevents the wash material from entering concentrate tank 2840. The wash material can then pass through the acoustophoretic device 2810 and remove the first media (e.g., bioreactor serum or preservative material). The wash material then exits via waste port 2608 and flows to waste tank 2850. The target particles remain trapped in the acoustophoretic device 2810.

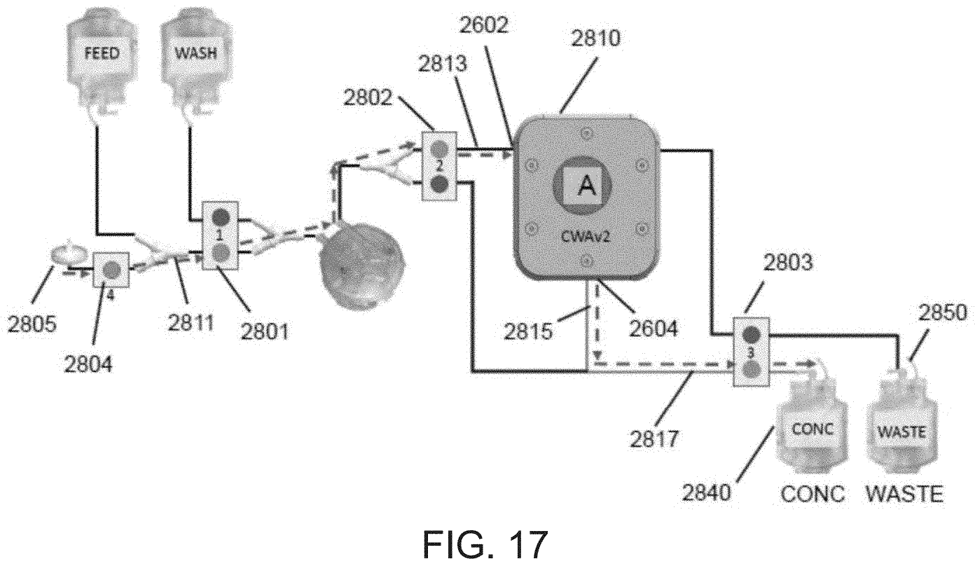

FIG. 17 illustrates the draining of the system (e.g., the collection of the target particles). Air intake valve 2804 is opened. The feed selector valve 2801 is operated with the bottom open (and top closed), and the inflow selector valve 2802 is operated with the top open (and bottom closed), so that air enters the acoustophoretic device 2810 via feed port 2602. The air generally aids in dislodging the clusters of target particles from the acoustophoretic device 2810. The outflow selector valve 2803 is operated with the bottom open (and top closed). The target particles flow out of wash/drain port 2604 through common line 2815, through drain line 2817 and subsequently to concentrate tank 2840.

Concentrating and washing cell culture is useful for producing biological products for industrial use. The systems of the present disclosure can be continuously improved and scaled up for handling of larger volumes.

In some examples, the acoustophoretic devices of the present disclosure may have a concentrated volume ranging from about 25 mL to about 75 mL. The devices may have a total cell capacity of about 4 billion to about 40 billion cells, or from about 4 billion to about 8 billion cells, or from about 20 billion to about 40 billion cells, or from about 16 billion to about 35 billion cells. The fluids entering and exiting the acoustophoretic devices may have cell densities from about 160 million cells/mL to about 670 million cells/mL, or from about 160 million cells/mL to about 320 million cells/mL, or from about 260 million cells/mL to about 535 million cells/mL, or from about 305 million cells/mL to about 670 million cells/mL, or from about 0.5 million cells/mL to about 5 million cells/mL.

The following examples are provided to illustrate the devices and processes of the present disclosure. The examples are merely illustrative and are not intended to limit the disclosure to the materials, conditions, or process parameters set forth therein.

### EXAMPLES

The ability of an acoustophoretic system of the present disclosure to concentrate Jurkat T-cells was tested. Jurkat T-cells have a diameter of 11 micrometers (μm) to 14 μm. An acoustophoretic device was used, and a Beckman Coulter Vi-CELL X was used at various test conditions to measure the cell density reduction.

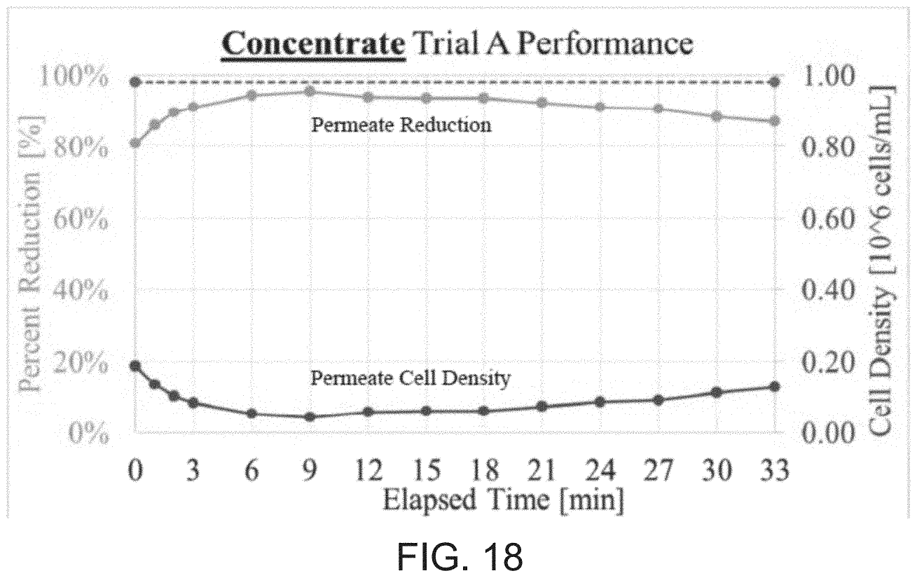

In the first trial A, the T-cells were concentrated, and the cell density of the permeate was measured. The dotted line indicates the feed cell density. Desirably, the cell density in the permeate is as low as possible, indicating that the cells are retained in the concentrate. The graph in FIG. 18 shows the results of trial A over time. The results show very low cell densities in the permeate, between 0.0 and 0.2 million cells/mL, showing that most of the cells are in the concentrate. The results also show a high permeate reduction percentage, between 80% and 99%.

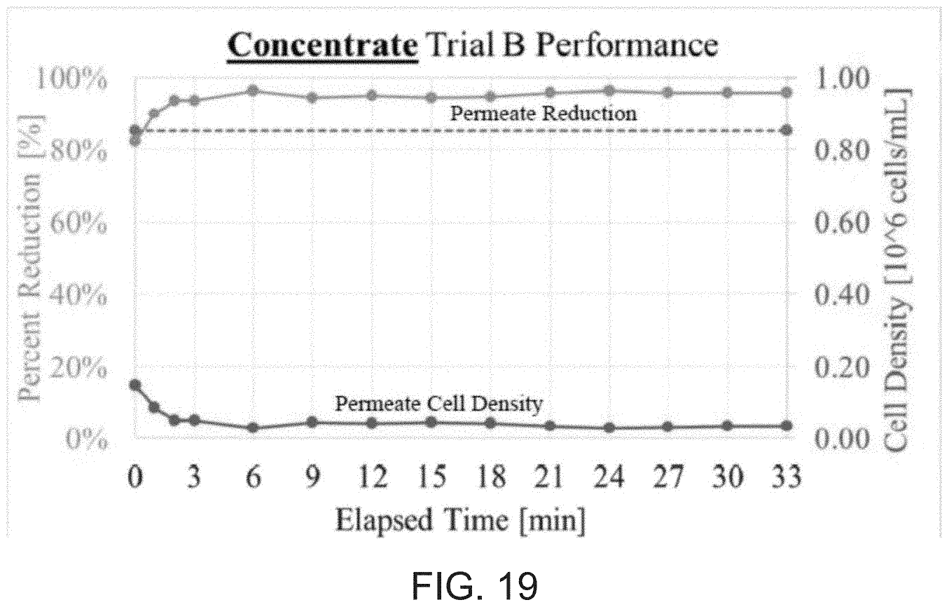

In the second trial B, the T-cells were concentrated, and the cell density of the permeate was measured. The dotted line indicates the feed cell density. FIG. 19 shows the results over time. The results show good performance, with the permeate cell density being below 0.1 million cells/mL after minute 1, and greater than 95% permeate reduction after minute 2.

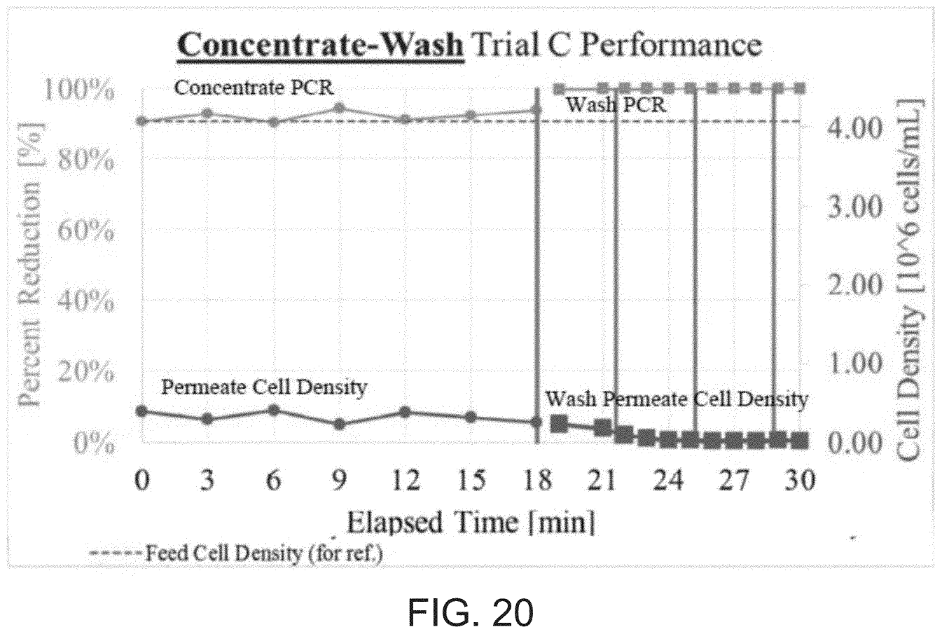

In the third trial C, the T-cells were concentrated and washed. The concentrating occurred for the first 18 minutes, and washing was subsequently performed. FIG. 20 shows the results over time. The dotted line indicates the feed cell density. The solid vertical lines indicate when concentrated system volumes were processed (three total volumes were processed). Note that this graph includes data on the concentrate and the permeate (not just the permeate). All of the cells obtained from concentration were maintained through washing, e.g., concentrated cells were not lost due to the addition of the washing process. Table 1 below provides additional information on these three trials. Retention and recovery rates of greater than 90% were obtainable for Jurkat T-cells.

TABLE 1

| | Feed | Feed | Concen- | | Concen- | Process |
|---|---|---|---|---|---|---|
| | Volume | Density | trate | Cell | tration | Time |
| Trial | (mL) | (cells/mL) | Volume | Recovery | Factor | (min) |
| A | 997 | 0.98 × 10⁶ | 21 mL | 91% | 47X | 33 |
| B | 1004 | 0.85 × 10⁶ | 21 mL | 95% | 48X | 33 |
| C | 555 | 4.08 × 10⁶ | 20 mL | 92% | 28X | 31 |

The liquid volumes used to completely wash the concentrated cells were tracked. Tracking the liquid volumes can be useful in applications such as, for example, removing electroporation buffer from a cell culture prior to transduction or transfection of the cell culture.

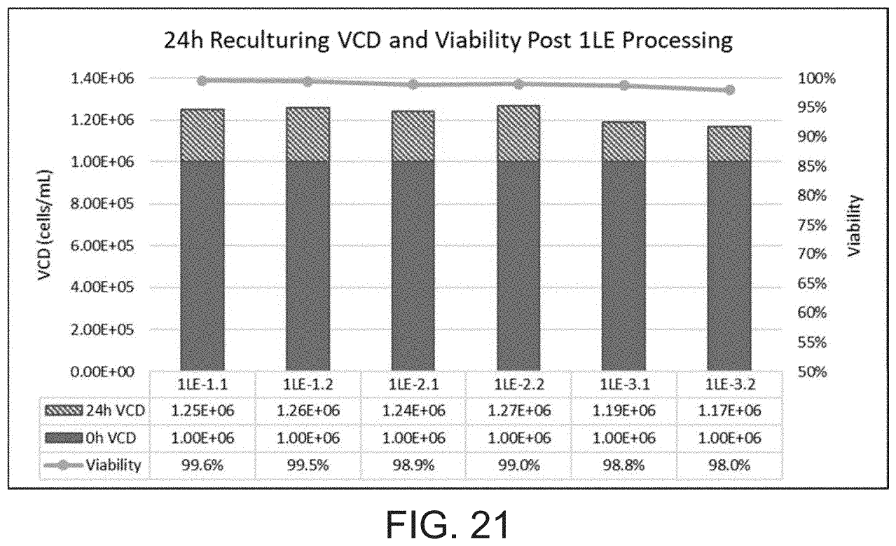

Table 2 below shows the input, output and performance experimental values for the low viable cell density (“1LE”, 1-5 E6mL-1) acoustic concentrate wash (ACW) volume reduction. FIG. 21 depicts the viable cell density (VCD) and viability of primary cultures of T-cells after 1LE processing. The cells from each 1LE experiment were re-seeded at 1E6mL-1, 37° C., 5% CO2 in duplicate and counted 24 h later.

TABLE 2
Low Cell Density 1LE ACW

| Experimental values | | |
|---|---|---|
| 1LE-1 | 1LE-2 | 1LE-3 |

Process
inputs

| Volume | 1105.8 | mL | 1092.2 | mL | 1102.6 | mL |
|---|---|---|---|---|---|---|
| Viable Cell | 1.86 | M/mL | 1.78 | M/mL | 2.49 | M/mL |
| Density | | | | | | |
| Total Viable | 2.1 | B | 1.9 | B | 2.8 | B |
| Cells | | | | | | |
| Cell viability | 99.1% | | 99.4% | | 99.3% | |
| Process | | | | | | |
| Outputs | | | | | | |
| Volume | 6.9 | mL | 5.8 | mL | 5.9 | mL |
| Viable Cell | 250.7 | M/mL | 243.3 | M/mL | 356.7 | M/mL |
| Density | | | | | | |
| Total Viable | 1.7 | B | 1.4 | B | 2.1 | B |
| Cells | | | | | | |
| Cell viability | 97.9% | | 98.0% | | 98.6% | |
| Process | | | | | | |
| performance | | | | | | |
| Viable Cell | 84% | | 73% | | 75% | |
| Recovery | | | | | | |
| Volume | 160-fold | | 188-fold | | 187-fold | |
| Reduction | | | | | | |
| Factor | | | | | | |
| Cell Concen- | 135-fold | | 137-fold | | 143-fold | |
| tration Factor | | | | | | |
| Process Time | 51 | min | 51 | min | 53 | min |

| | | | |
|---|---|---|---|
| Wash | n/a | n/a | n/a |
| Residuals | | | |

All the tests were performed according to the specifications, yielding a concentrate volume between 5 and 7 mL in under an hour. The processed volumes were about 1100 ml with cell densities between 1.8 and 2.5 E6 ml-1. The total number of cells processed ranged from 1.9 to 2.8 B cells with cell viability exceeding 99%. After concentration, the collected cell concentration volume varied from 5.8 to 6.9 ml with cell densities between 243 and 357 E6 ml-1. This represents a cell recovery of 1.4 to 2.1 B cells with no noticeable drop in cell viability. The viable cell recovery was therefore between 73 and 84%, which constitutes a volume reduction factor between 160 and 187 and a cell density concentration factor ranging from 135 to 143. The process times was about 50 min.

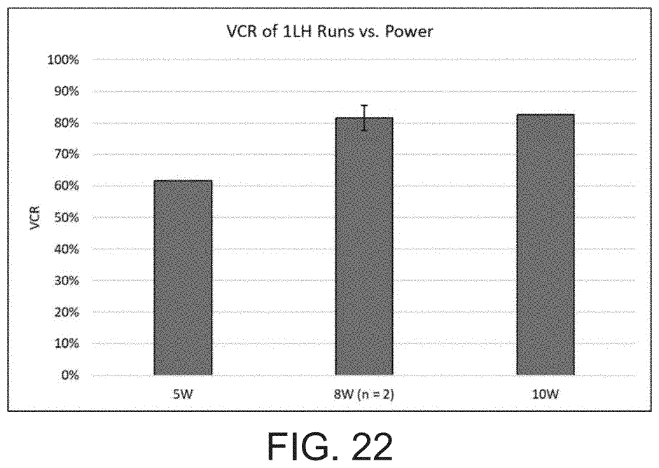

Table 3 displays the input, output and performance experimental values for the high viable cell density (“1LH”, 10-40 E6mL-1) ACW volume reduction. FIG. 22 shows the effect of power on Viable Cell Recovery (VCR) using the high cell density 1LH element at a flow rate of 30 mL/min.

TABLE 3
High Cell Density 1LH ACW

| Experimental values | | | |
|---|---|---|---|
| 1LH-1 (5 W) | 1LH-2 (8 W) | 1LH-3 (8 W) | 1LH-4 (10 W) |

Process
inputs

| Volume | 976.2 | mL | 949.9 | mL | 931.9 | mL | 908.6 | mL |
|---|---|---|---|---|---|---|---|---|
| Viable Cell | 39.0 | M/mL | 35.3 | M/mL | 32.3 | M/mL | 31.4 | M/mL |
| Density | | | | | | | | |
| Total Viable | 38.1 | B | 33.5 | B | 30.1 | B | 28.6 | B |
| Cells | | | | | | | | |
| Cell viability | 97.9% | | 98.8% | | 98.8% | | 98.2% | |
| Process | | | | | | | | |
| Outputs | | | | | | | | |
| Volume | 49.9 | mL | 48.9 | mL | 48.4 | mL | 48.9 | mL |
| Viable Cell | 470 | M/mL | 587 | M/mL | 483 | M/mL | 483 | M/mL |
| Density | | | | | | | | |
| Total Viable | 23.5 | B | 28.7 | B | 23.4 | B | 23.6 | B |
| Cells | | | | | | | | |
| Cell viability | 99.0% | | 97.6% | | 97.7% | | 97.5% | |
| Process | | | | | | | | |
| performance | | | | | | | | |
| Viable Cell | 62% | | 86% | | 78% | | 83% | |
| Recovery | | | | | | | | |
| Volume | 20-fold | | 19-fold | | 19-fold | | 19-fold | |
| Reduction | | | | | | | | |
| Factor | | | | | | | | |
| Cell Concen- | 12-fold | | 17-fold | | 15-fold | | 15-fold | |
| tration Factor | | | | | | | | |
| Process Time | 31 | min | 33 | min | 36 | min | 35 | min |

| | | | | |
|---|---|---|---|---|
| Wash | n/a | n/a | n/a | n/a |
| Residuals | | | | |

Processed volumes were about 950 ml with viable cell densities from 31 to 39 E6 ml-1 corresponding to a range of cell numbers between 29 and 38 B cells at cell viabilities exceeding 98%. Cell concentrate volumes averaged 49 ml. Process times were between 31 and 36 min.

The 5 W test yielded a low cell recovery of 62%. The increase to 8 W increased the performance to the desired range (cell recovery of 78 to 86%), whereas a power increase to 10 W did not seem to improve the cell recovery (83%). While further replicates are required at each power level to provide definitive conclusions, these results suggest that powers above 8 W are required to have a VCR near and above 80%. Volume reduction factors were about 19 and cell concentrations factors ranged from 12 (5 W) to 17 (8 W). The final cell concentrations obtained during the 1LH experiments, 470-590 E6mL-1, are comparable to the pellet concentrations in dead-end centrifugation processes for suspension cell lines (200-600 e6mL-1.

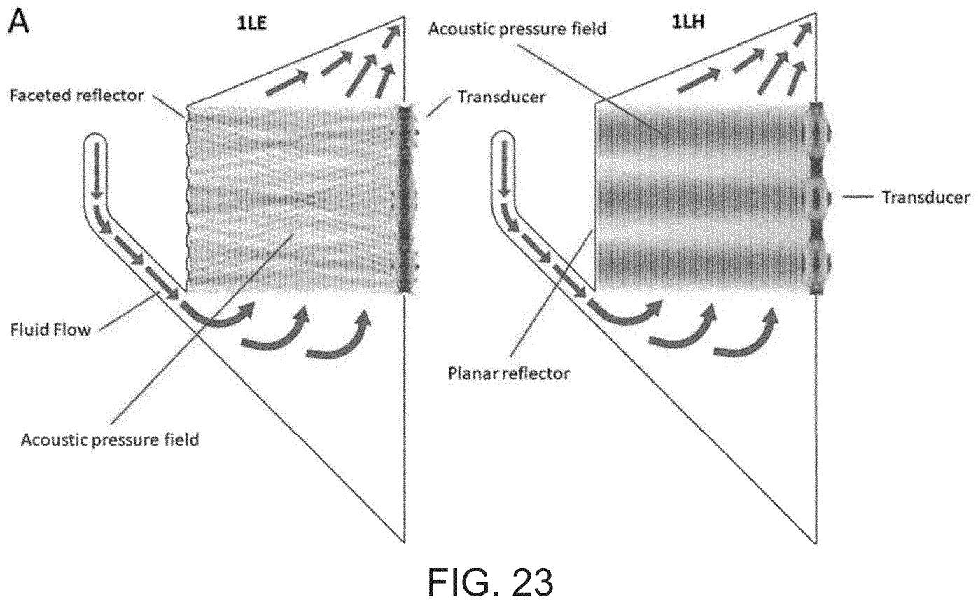

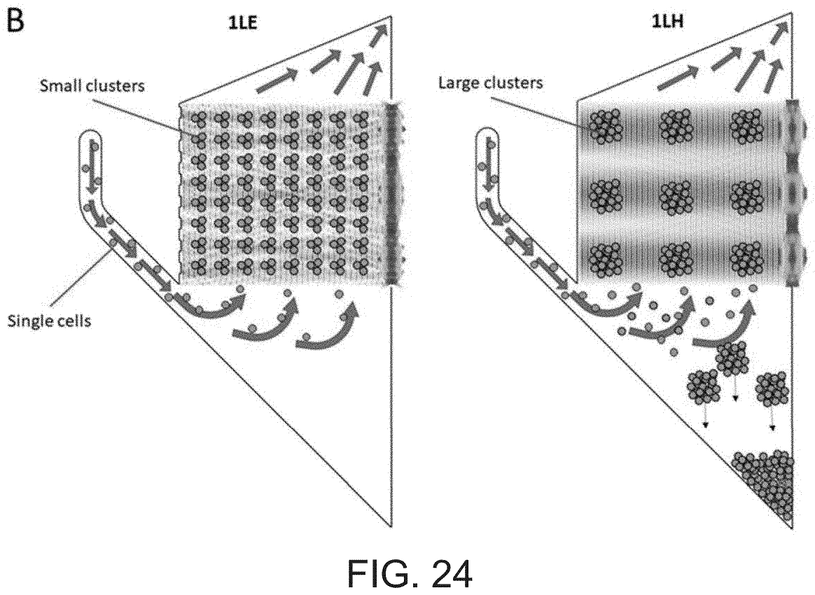

FIG. 23 shows two typical acoustic standing wave fields and the process flow diagram for a low cell concentration concentrate/wash application (1LE) and for a high cell density concentrate/wash application (1LH). In each scenario, the piezoelectric transducer is on the right and is excited in one of its multimodal displacement patterns. For the 1LH a flat planar reflector is used, whereas for the 1LE a faceted reflector is used. The flat planar reflector combined with the multimode of the transducer sets up a multidimensional standing wave resulting in three parallel standing waves with strong lateral amplitude gradients. The acoustic radiation force is proportional to the gradient of the acoustic pressure amplitude. This setup therefore has sufficient trapping potential to trap cells, but also to generate cell clusters of sufficient size resulting in a continuous gravitational settling of these cell clusters. This is shown schematically in FIG. 24 on the right. This continuous separation of cells is useful when performing a concentration/wash unit operation of a high cell density cell culture, e.g., the cells being removed from the acoustic standing wave field. On the other hand, for small cell density cell cultures, the total number of cells to be processed are such that all the cells can be trapped and held in the acoustic standing wave. Therefore, the goal is to maximize the trapping potential of the standing wave field. This goal can be achieved by the use of a faceted reflector. These facets reflect and scatter the acoustic wave, generating more and stronger acoustic radiation potential wells where cells will be trapped, see FIG. 23, left, 1LE, and form smaller clusters which do not settle while the acoustic field is active, or until the acoustic field is turned off.

Multidimensional Acoustic Concentrate-Wash (ACW) for low cell concentration is shown in FIGS. 23, 24 (left side device, 1LE, 1-5E6 cells mL-1) and high cell concentration (right side device, 1LH, >10E6 cells mL-1). The cross-sectional views of the design represent the acoustic pressure field established in 1 LE and 1LH devices and the transducer displacement profile and the principle of trapping and cluster formation to achieve cell separation. The devices include a collector drain in the bottom (not shown).

During the start-up portion of the concentration/wash unit operations, a recirculation is used to generate the initial clusters in the standing wave, e.g., performing a seeding of clusters. Once the initial clusters are formed, the trapping efficiency increases due to secondary acoustic radiation forces, whereby the larger clusters exert an attractive force on the incoming cells.

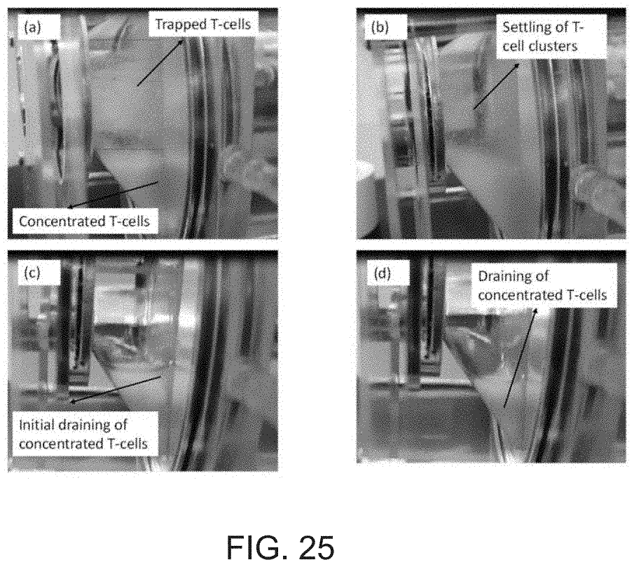

FIG. 25 shows photographs of various stages of the T-cell concentration process in the ACW device: (a) trapping of T-cells in the acoustic field and settled out T-cells in the collector, (b) settling of T-cell clusters after acoustic field is turned off, (c) initial draining of T-cells from collector, and (d) final stage of draining of T-cells from the collector. The Viable Cell Recovery (VCR (%)) is calculated according to the balance of viable cells present in the concentrate relative to the feed (see derivation below). Wash residuals were not calculated for these trials, as the focus of these experiments was to demonstrate concentration efficiency.

$$VCR ( \% ) = \frac{Viable Cells Concentrated}{Viable Cells Processed} \times 100 = \frac{ Concentrate Volume \times {VCD}_{c o n c e n t r a t e}}{Processed Feed Volume \times {VCD}_{feed}} \times 100$$

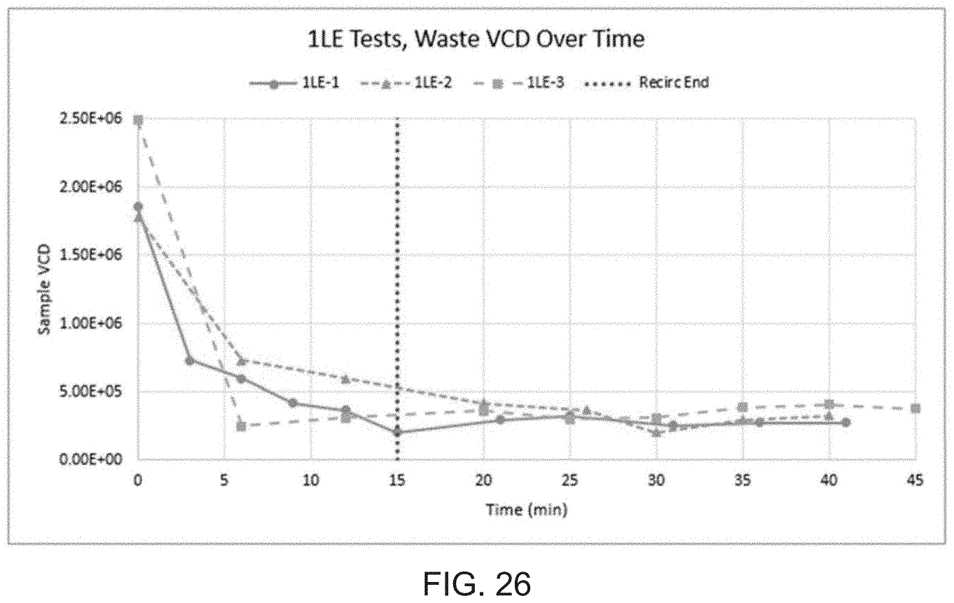

The 1LE application was performed in triplicate (experiments designated as 1LE-1, -2, and -3) using primary cultures of T-cells and fixed process parameters. The acoustic elements were assembled, double-bagged and gamma irradiated to perform the concentration in an aseptic environment. The process parameters were a nominal flow rate of 30 mL/min (˜2 L/hr) and acoustic drive conditions of 2.24 MHz/40 W per channel. The recirculation period is required before the concentration step because the 1LE system's efficiency is enhanced by the number of cells that have populated the acoustic standing wave. Recirculating the waste back to the feed allows cells to enter the system and be retained by the acoustics, gradually building efficiency, without losing the initial unretained cells to the waste stream. Based on previous testing with Jurkat T-cells, the recirculation mode duration was selected to be 15 minutes because the system is approximately 80% efficient at that point for feeds in this cell density range. FIG. 26 depicts the waste VCD (E6 ml-1) versus time (min) for the three 1LE experiments (30 mL/min) with initial cell densities on the order of 2E6 ml-1 and demonstrates the usefulness of a 15-minute recirculation period to populate the 3D acoustic standing wave (at higher cell densities such as 1LE-3 and with the same flow rate the acoustic standing wave will be populated faster).

The 1LH ACW was operated at different powers to assess the effect on viable cell recovery. Four tests were performed at the input feed specification using a fixed flow rate of 30 mL/min (˜2 L/hr). The first test was performed at a power level of 5 W. The second and third tests were performed at 8 W, and for the fourth test the power was increased to 10 W. The waste of this fourth test was reprocessed through the same element to simulate the operation of a two stage ACW (i.e. two acoustic elements in series). At higher feed cell densities, a significant reduction in transducer power is achieved, i.e., 5-10 W for 1LH versus 40 W for 1 LE, which eliminates the need for any cooling of the system.

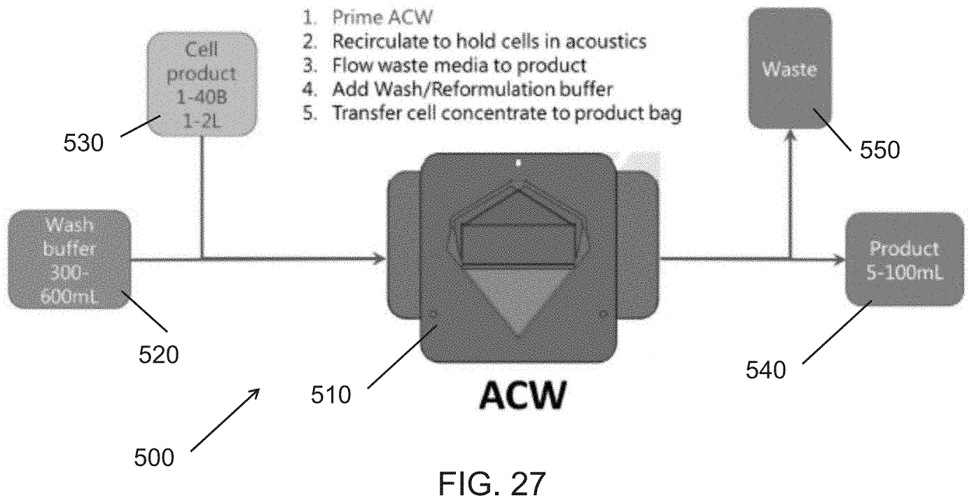

FIG. 27 shows a process flow diagram for operations in a T-cell concentration and wash system 500. A typical process time for the concentrate and wash operation ranges from 60 to 90 minutes. Acoustic concentrate wash (ACW) device 510 has inputs of a cell product 530 and a wash buffer 520. Wash buffer 520 may have a volume in a range of from about 300 mL to about 600 mL. Cell product 530 may consist of 40 billion cells in a 2 L volume. ACW device 510 has outputs of a product 540 and waste 550. Product 540 may have a volume range of from about 5 to about 100 mL. Waste 550 may consist of stray cells, cell debris and buffer solution.

Operation of ACW device 510 is implemented with the protocol as illustrated in FIG. 27. The operation begins with ACW device 510 being primed, such as by flowing cell product 530 into an internal acoustic chamber of ACW device 510. The cellular material in cell product 530 tends to build up within the acoustic chamber during the priming stage to permit ACW device 510 to operate with greater efficiency. The output of ACW device 510 can be recirculated to the input (not shown). Accordingly, as cells are being trapped in the acoustic wave in the acoustic chamber, and trapping efficiency increases, cells that pass through the acoustic chamber can be returned to the input so as not to lose cellular content while ACW device 510 is being primed.

Once ACW device 510 is primed, the recirculation path is closed, and additional cell product 530 is flowed to the acoustic chamber. As cell product 530 flows into the primed acoustic chamber additional cells are captured in the acoustic wave, while the media flows out of the acoustic chamber to waste 550.

Once all the cells are collected in the acoustic wave in the acoustic chamber, wash buffer 520 is flowed into the acoustic chamber, which causes the previous media in the acoustic chamber to be washed out, and directed to waste 550. At that point, the cells are washed and concentrated, and can be removed from the acoustic chamber and provided to product 540.

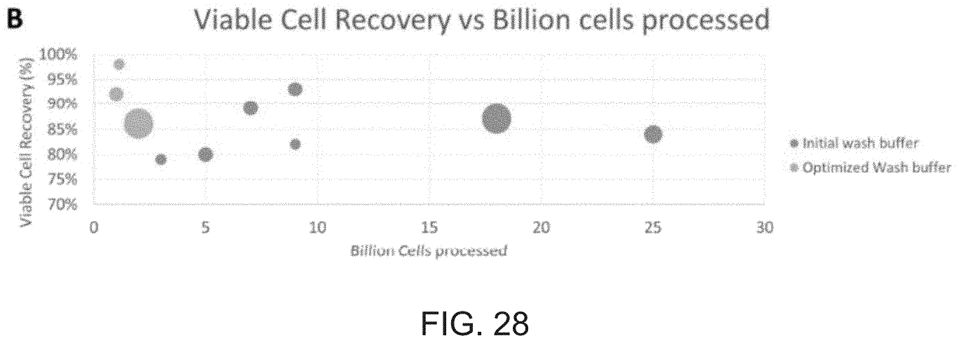

FIG. 28 is a bubble graph showing viable cell recovery versus billions of cells processed. The total viable cells processed is plotted on the x-axis and the corresponding viable cell recovery is plotted on the y-axis. The size of the bubbles in the bubble graph represent the processed volume in a range of from about 0.75 to about 2 L. The bubble graph results are for a concentrate wash operation with Jurkat T-cells with a processing time of between 60 to 90 minutes. 1 billion cells in 1 L of media were processed, yielding a 92+/−3% viable cell recovery and in output volume of 5-6 mL.

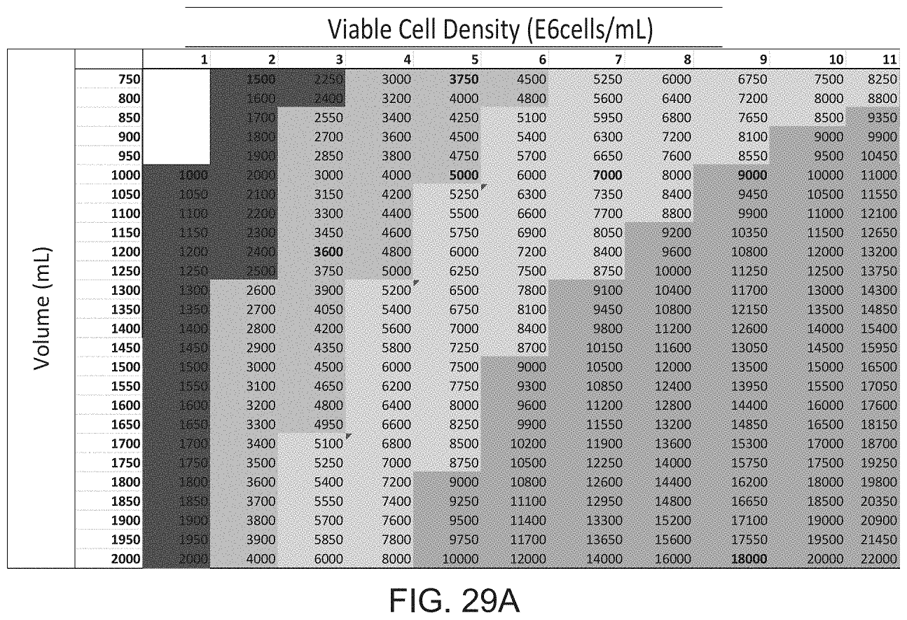

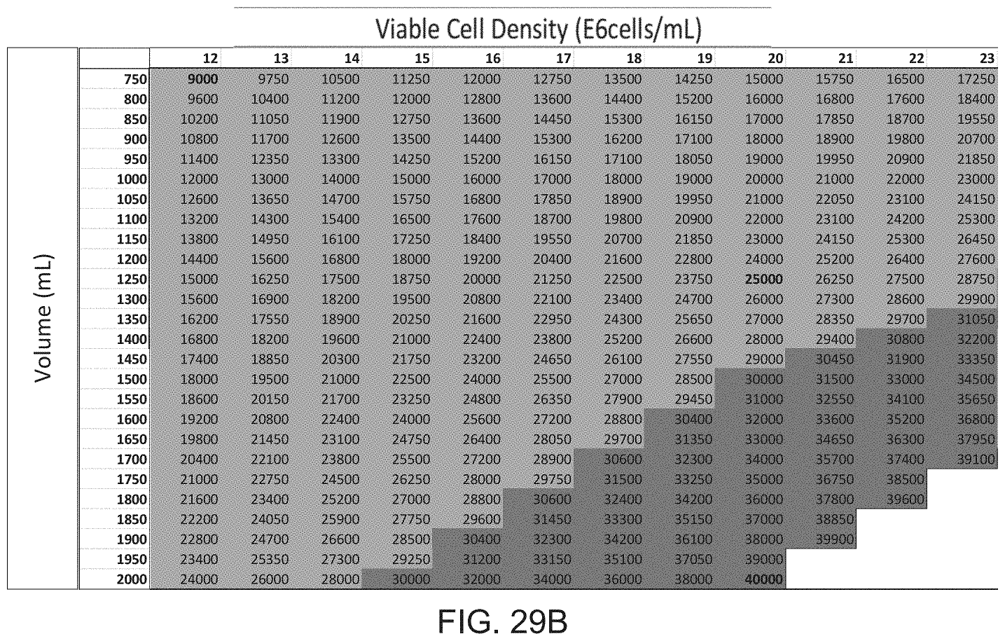

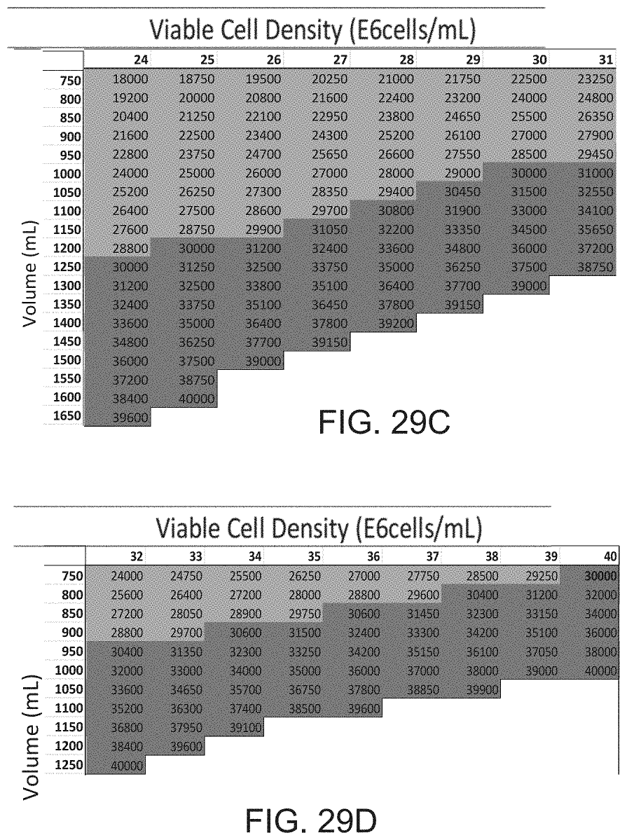

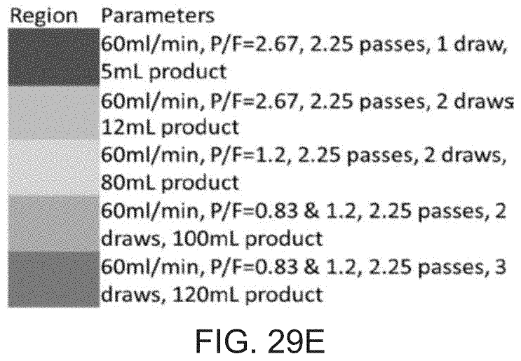

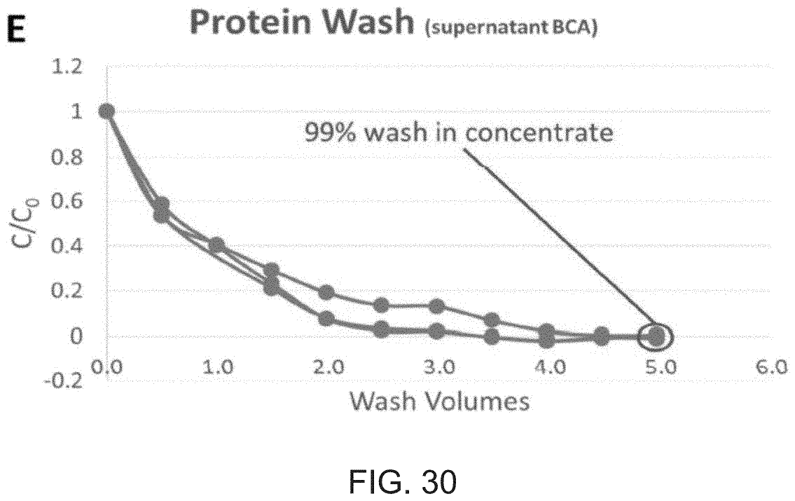

FIGS. 29A, 29B, 29C, 29D, and 29E illustrate a process map for acoustic concentrate wash operations such as those illustrated in FIGS. 27, 28. The process map charts operational parameters of acoustic power, flow rate, number of cell collection cycles, number of cells and cell concentration. FIG. 30 is a graph showing total protein concentration reduction in the permeate, as a function of chamber volumes of wash buffer. After five chamber volumes are passed through the cells, the final total protein content is reduced by 99%.

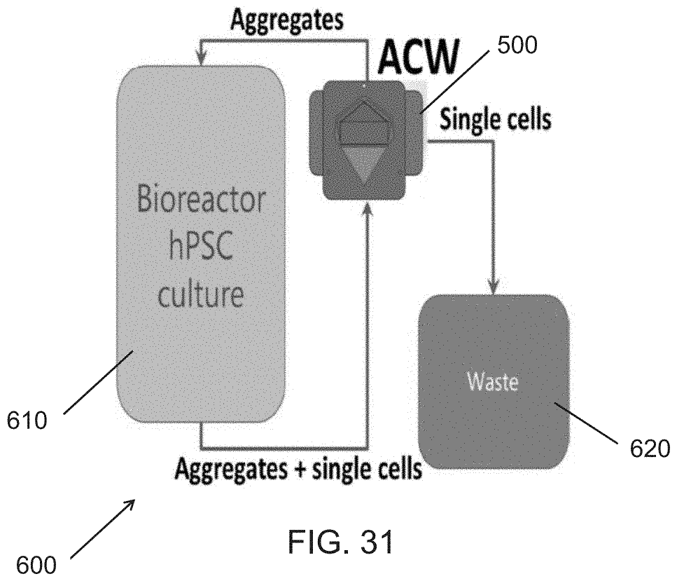

FIG. 31 shows an application of ACW device 500 for removal of single cells from cellular aggregates in a bioreactor 610. Human pluripotent stem cells (hPSCs) can aggregate within bioreactor 610, which process may be somewhat inefficient. Such a process may lead to the generation of a population of single cells that may affect the growth and differentiation of hPSC aggregates as well as final product quality. ACW device 500 can be used to remove such single cells while retaining the cellular aggregates in the bioreactor. In this application, the aggregates and single cells are provided to ACW device 500, where the larger aggregates are trapped in the acoustic standing wave in the acoustic chamber of ACW device 500. The smaller single cells pass through the acoustic wave and exit the acoustic chamber to waste 620. The larger aggregates can be collected in the acoustic wave and returned to bioreactor 610. Over time, single cells are removed from bioreactor 610 while aggregates are maintained, thereby concentrating aggregates and depleting single cells in bioreactors 610.

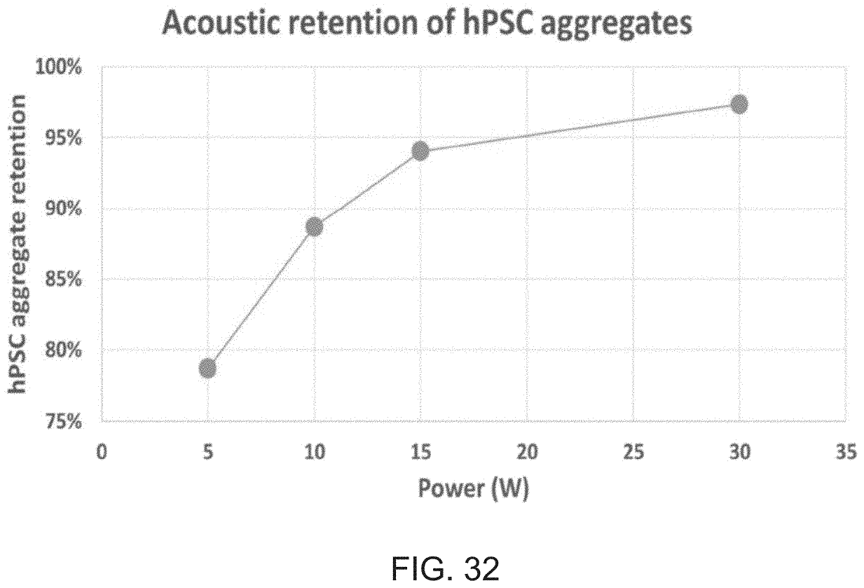

FIG. 32 is a graph showing hPSC retention versus acoustic power. In the graph, acoustic power was modulated between from about 5 W to about 30 W. As shown in the graph, the greatest aggregate retentions occurred at 30 W. Inspection of the retained aggregates show that their morphology was not changed during processing in ACW device 500.

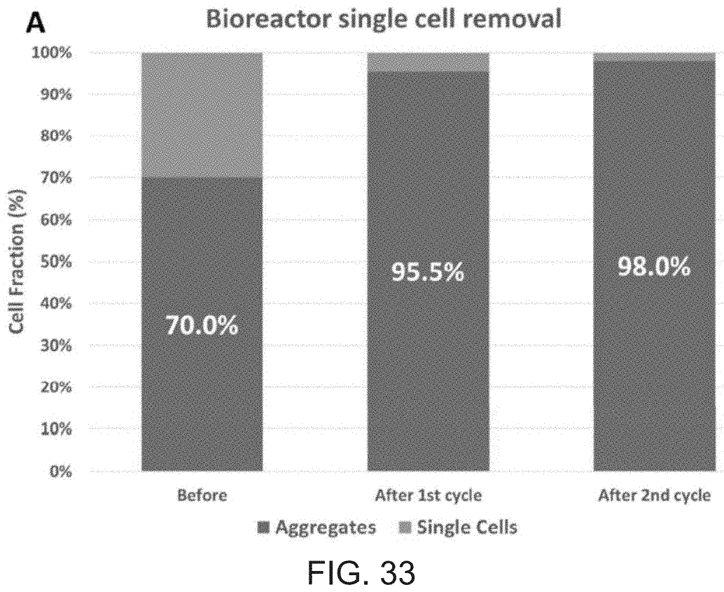

FIG. 33 is a bar graph showing single cell removal from an aggregate population in a bioreactor. A 2 L bioreactor was connected to ACW device 500 and run at 60 mL per minute at 30 W with aggregates returned to the bioreactor every five minutes. The bar graph illustrates the increasing fraction of aggregates held in the bioreactor as the bioreactor contents were processed through ACW device 500. Inspection of the retained aggregates indicated that the initial morphology remained the same after acoustic processing. The total aggregate recovery was 85%.

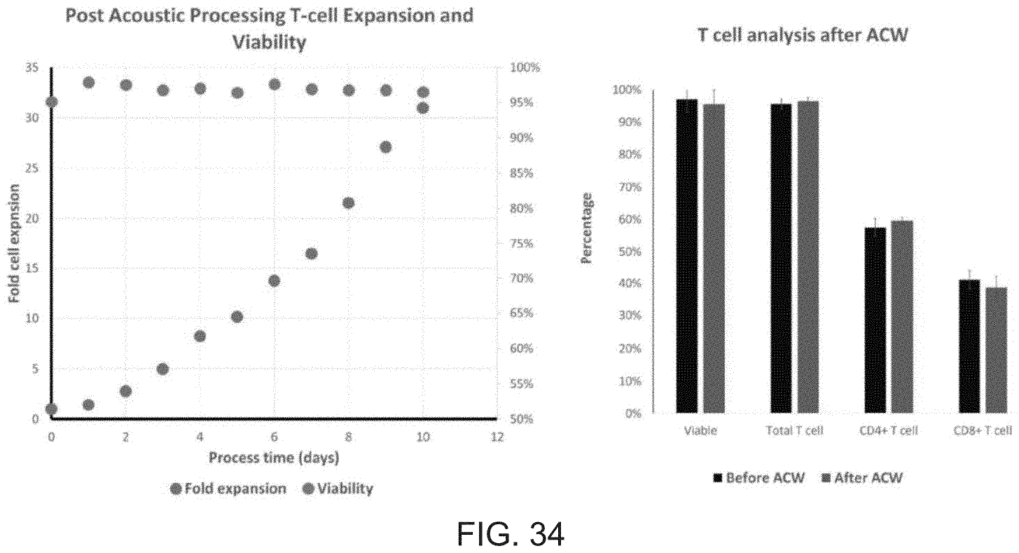

FIG. 34 shows two graphs for acoustic concentrate wash processing, one charting cell expansion versus process time (left side) and one charting changes in T-cell population after acoustic concentrate wash processing. A primary culture of human CAR-T cells processed with the acoustic concentrate wash device grow at their typical growth rate (30 h doubling time). The cell viability is maintained at a high level and a 30-fold expansion can be realized in a perfused Xuri bioreactor culture (left graph). The right graph illustrates an n=2 donors comparison of T-cell viability and phenotype before and after processing with the acoustic concentrate wash device. No significant changes are observed in the cell surface marker expression after processing by the acoustic concentrate wash device.

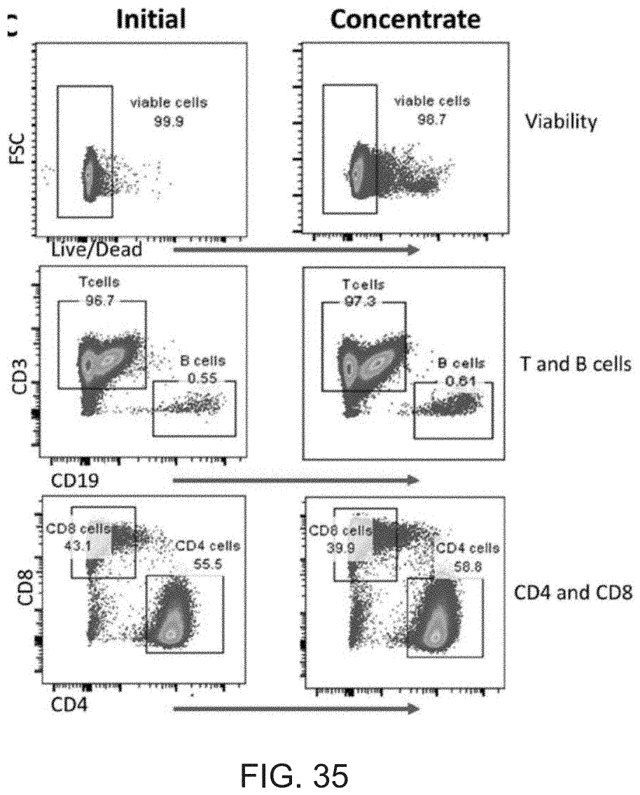

FIG. 35 illustrates a representative flow cytometry plot depicting typical T-cell surface markers, whose expression remains unchanged after acoustic concentrate wash processing. The processing provides a 90% average cell recovery and 99% protein wash with an input volume of 1-2 L and an output volume of 5-120 mL. An output VCD of 100-500 M/mL is obtained with an input VCD of 1-40 M/mL. No loss in viability was observed, the percentage of T-cells was unchanged, and the ratio of CD8/CD4 remained unchanged.

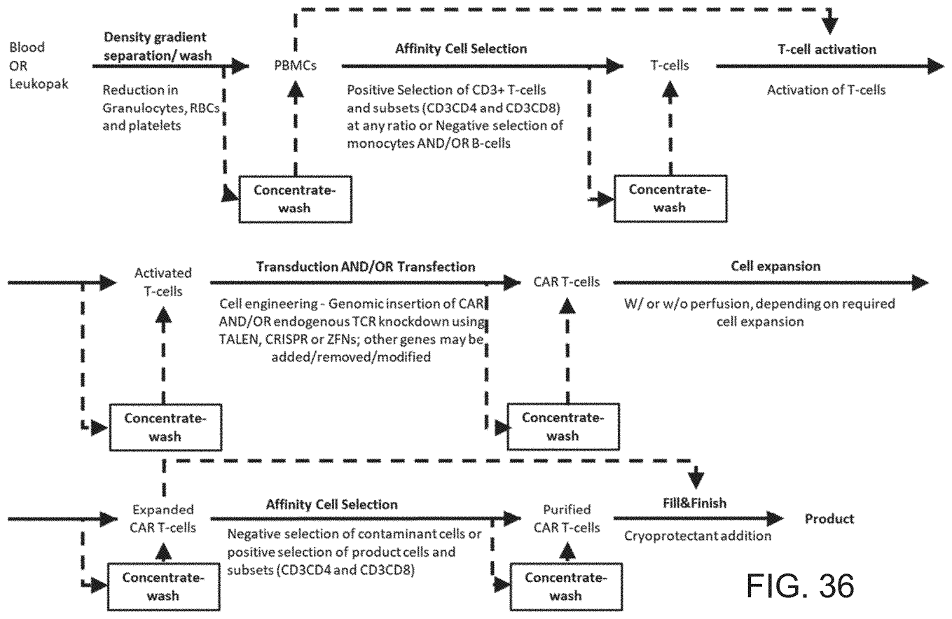

FIG. 36 illustrates an end to end cell therapy production process using the concentrate wash device of the present disclosure at a number of points in the process. The concentrate wash device may be used to reduce granulocytes, RBCs and platelets in an original blood product. Following affinity cell selection, the concentrate wash device can be used to collect and wash the resulting T-cells. Following T-cell activation, the T-cells can again be concentrated and washed prior to a transduction/transfection step. The concentrate wash device may be employed after the transduction/transfection step, prior to cell expansion. Following cell expansion, the concentrate wash device can be used to collect and wash the expanded CAR-T cells. Following an affinity cell selection step two remove contaminants cells or select desired cells, another concentrate wash process can be employed.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

1. A method of washing particles, the method comprising:
obtaining an initial particle density and volume for an initial mixture of a first media and particles;
setting the parameters of an acoustic concentrate wash process using an acoustophoretic device based on the initial particle density and volume of the initial mixture;
providing the initial mixture to a chamber of the acoustophoretic device, the acoustophoretic device including at least one ultrasonic transducer that includes a piezoelectric material;
driving the at least one ultrasonic transducer to create an acoustic wave in the chamber, such that at least a portion of the particles are retained in an acoustic field generated at least in part by the acoustic wave; and
operating the acoustophoretic device in accordance with the parameters.

2. The method of claim 1, further comprising providing a second media to the chamber that is a biocompatible wash or a buffer solution.

3. The method of claim 1, wherein the particles are cells.

4. The method of claim 1, wherein the particles are microcarrier/cell complexes.

5. The method of claim 1, wherein the initial mixture has a density of about 0.5 million particles/mL to about 5 million particles/mL.

6. The method of claim 1, further comprising concentrating the particles in the initial mixture.

7. The method of claim 1, further comprising concentrating the particles to a concentrate volume that is about 25 to about 50 times less than a volume of the initial mixture.

8. The method of claim 7, further comprising concentrating the particles in the initial mixture to a concentrated particle density of about 25 to about 50 times greater than a particle density of the initial mixture.

9. The method of claim 1, wherein a cell density of a wash output of the flow chamber is about 0.0 to about 0.5 million cells/mL.

10. The method of claim 9, wherein the wash output is from a concentrate process and a wash process.

11. The method of claim 1, further comprising conducting a spectrophotometer process on the chamber to determine wash efficacy.

12. A method of recovering cells from a cell culture, comprising:
obtaining an initial particle density and volume for an initial mixture of a first media and particles;
setting the parameters of an acoustic concentrate wash process using an acoustophoretic device based on the initial particle density and volume of the initial mixture;
feeding the initial mixture of the cell culture to a flow chamber of the acoustophoretic device, the acoustophoretic device including at least one ultrasonic transducer that includes a piezoelectric material that is configured to be driven to generate a multi-dimensional acoustic wave in the flow chamber; and
driving the at least one ultrasonic transducer to generate a multi-dimensional acoustic wave in the flow chamber;
retaining the cells from the initial mixture in an acoustic field generated at least in part by the multi-dimensional acoustic wave to concentrate the cells;
operating the acoustophoretic device in accordance with the parameters.

13. The method of claim 12, wherein the cell density of the concentrated cells is about 25 to about 50 times greater than the cell density of the initial mixture.

14. The method of claim 12, wherein a volume of the concentrated cells is 25 to about 50 times less than a volume of the initial mixture.

15. The method of claim 12, wherein the concentrated cells are obtained in about 35 minutes or less.

16. The method of claim 12, further comprising washing the concentrated cells, wherein a cell density of a wash output of the flow chamber is about 0.0 to about 0.5 million cells/mL.

17. A concentrate wash system, comprising:
a pump;
a plurality of valves;
a flow chamber;
at least one ultrasonic transducer coupled to the flow chamber and including a piezoelectric material that is adapted to be driven to generate a multi-dimensional acoustic wave; and
a controller for controlling the pump, plurality of valves and at least one ultrasonic transducer, the controller being configurable with parameters that include one or more of acoustic power, flow rate, number of cell collection cycles, number of cells or cell concentration.

18. The concentrate wash system of claim 17, wherein the flow chamber further comprises a volume of about 25 mL to about 75 mL.

19. The concentrate wash system of claim 18, wherein the flow chamber can contain a cell capacity of about 4 billion to about 40 billion cells.
